(19) United States
(12) Patent Application Publication
(10) Pub. No.: US 2013/0197288 A1
(43) Pub. Date: Aug. 1, 2013

(54) PROCESS FOR THE CONVERSION OF SYNTHESIS GAS TO OLEFINS

(71) Applicants: BASF SE, Ludwigshafen (DE); Linde AG, Pullach (DE)

(72) Inventors: Alexander Schäfer, Limburgerhof (DE); Kirsten Spannhoff, Mannheim (DE); Ekkehard Schwab, Neustadt (DE); Christian Thaller, Munchen (DE); Harald Schmaderer, Wolfratshausen (DE); Nicole Schödel, Munchen (DE); Ernst Haidegger, Riemerling (DE); Holger Schmigalle, Wolfratshausen (DE); Axel Behrens, Munchen (DE); Volker Göke, Geretsried (DE)

(73) Assignees: Linde AG, Pullach (DE); BASF SE, Ludwigshafen (DE)

(21) Appl. No.: 13/753,661

(22) Filed: Jan. 30, 2013

Related U.S. Application Data
(60) Provisional application No. 61/718,210, filed on Oct. 25, 2012.

(30) Foreign Application Priority Data
Jan. 31, 2012 (DE) 10 2012 001 802.9

(51) Int. Cl. C07C 1/04

(52) U.S. Cl. CPC C07C 1/0425
USPC 585/324

(57) ABSTRACT

The present invention relates to a process for converting a gas mixture comprising CO and H₂ to olefins, comprising
(1) providing a gas mixture (G0) comprising CO and H₂;
(2) providing a catalyst (C1) for conversion of CO and H₂ to dimethyl ether;
(3) contacting the gas mixture (G0) with the catalyst (C1) to obtain a gas mixture (G1) comprising dimethyl ether and CO₂;
(4) providing a catalyst (C2) for conversion of dimethyl ether to olefins;
(5) contacting the gas mixture (G1) comprising dimethyl ether with the catalyst (C2) to obtain an olefin-comprising gas mixture (G2).

The present invention relates to a process for conversion of a gas mixture comprising CO and H₂ to olefins using a first catalyst for conversion of CO and H₂ to dimethyl ether, and the gas mixture formed therefrom is converted to the olefinic product using a second catalyst for conversion of dimethyl ether to olefins. The present invention also relates to a process for preparing olefins from carbon or hydrocarbon.

### INTRODUCTION

In view of increasing scarcity of mineral oil deposits which serve as starting material for preparation of lower hydrocarbons and derivatives thereof, alternative processes for preparing such commodity chemicals are becoming increasingly important. In alternative processes for obtaining lower hydrocarbons and derivatives thereof, specific catalysts are frequently used in order to obtain lower hydrocarbons and derivatives thereof, such as unsaturated lower hydrocarbons in particular, with maximum selectivity from other raw materials and/or chemicals. In this context, important processes include those in which methanol as a starting chemical is subjected to a catalytic conversion, which can generally give rise to a mixture of hydrocarbons and derivatives thereof, and also aromatics.

In the case of such catalytic conversions, the particular challenge is to refine the catalysts used therein, and also the process regime and parameters thereof, in such a way that a few very specific products form with maximum selectivity in the catalytic conversion. Thus, these processes are named particularly according to the products which are obtained in the main therein. In the past few decades, particular significance has been gained by those processes which enable the conversion of methanol to olefins and are accordingly characterized as methanol-to-olefin processes (MTO process for methanol to olefins). For this purpose, there has been development particularly of catalysts and processes which convert the conversion of methanol via the dimethyl ether intermediate to mixtures whose main constituents are ethene and propene.

U.S. Pat. No. 4,049,573, for example, relates to a catalytic process for conversion of lower alcohols and ethers thereof, and especially methanol and dimethyl ether, selectively to a hydrocarbon mixture with a high proportion of C₂-C₃-olefins and monocyclic aromatics and especially para-xylene.

Goryainova et al. in Petroleum Chemistry 2011, volume 51, no. 3, p. 169-173 describes the catalytic conversion of dimethyl ether to lower olefins using magnesium-containing zeolites.

Starting materials which are used in such processes and especially methanol and dimethyl ether are frequently obtained by the reforming of natural gas, it being possible to integrate the reforming step into the process in order to obtain hydrocarbonaceous products from natural gas via a number of intermediates. In this context, mention should be made, for example, of the Haldor-Topsøe-TIGAS process, in which synthesis gas is first obtained from a mixture of natural gas, steam and oxygen by reforming, and this is then converted by catalytic reaction to methanol, which is finally converted in a methanol-to-gasoline process (MTG process) to gasoline-containing products (see, for example, F. J. Keil, Microporous and Mesoporous Materials 1999, volume 29, pages 49-66).

Lee et al. in Fuel Science and Technology International 1995, volume 13, pages 1039-1057 also describes the production of gasoline from synthesis gas, the intermediate obtained being not methanol but dimethyl ether. Thus, dimethyl ether is first obtained from synthesis gas, and is then converted catalytically to gasoline.

In spite of the advances which have been achieved with respect to the selection of raw materials and the conversion products thereof which can be used for the production of hydrocarbonaceous products, there is still a need for novel processes and catalysts which give a higher efficiency for the conversion. More particularly, there is a constant need for novel processes and catalysts which, proceeding from the raw materials, lead via a minimum number of intermediates very selectively to the desired end product. Furthermore, it is desirable for efficiency to be enhanced further by development of processes which require a minimum number of workup steps for the intermediates in order that they can be used in the subsequent stages.

### DETAILED DESCRIPTION

It was an object of the present invention to provide an improved process for obtaining olefins, which uses synthesis gas as the raw material. More particularly, it was an object of the present invention to provide, though the selective use of specific catalysts, a process which enables the conversion of synthesis gas to olefins, if at all possible without workup of the intermediates formed.

It has thus been found that, surprisingly, it is possible to run a process for converting synthesis gas to olefins which leads selectively via a mixture of dimethyl ether and CO₂. In this process, dimethyl ether can be supplied without further treatment, except for the optional removal of CO₂, to the synthesis stage for olefin preparation. The olefin synthesis is preferably effected without an intermediate removal of CO₂. In addition, it has been found that, unexpectedly, it is also possible to dispense with the addition of an inert gas between the individual process stages. Finally, it has been found that, surprisingly, the effect of the presence of CO₂ in a gas mixture which is obtained as an intermediate is that the coking and hence the deactivation of the downstream catalyst for conversion of dimethyl ether to olefins can be effectively suppressed owing to the Boudouard equilibrium. Thus, a highly efficient process for conversion of synthesis gas to olefins has been found, this not only significantly simplifying or even obviating the need for the purification of the intermediate formed, but quite unexpectedly also enabling even longer service lives of the catalyst with respect to a process in which such a purification is performed.

Thus, the present invention relates to a process for converting a gas mixture comprising CO and H₂ to olefins, comprising
(1) providing a gas mixture (G0) comprising CO and H₂;
(2) providing a catalyst (C1) for conversion of CO and H₂ to dimethyl ether;
(3) contacting the gas mixture (G0) with the catalyst (C1) to obtain a gas mixture (G1) comprising dimethyl ether and CO₂;
(4) providing a catalyst (C2) for conversion of dimethyl ether to olefins;
(5) contacting the gas mixture (G1) comprising dimethyl ether with the catalyst (C2) to obtain an olefin-comprising gas mixture (G2).

With regard to the gas mixture (G0) provided in (1), there is no restriction whatsoever in principle with respect to the composition thereof, provided that it allows the conversion of at least some of the CO and H₂ present therein to dimethyl ether and CO₂ in step (3). This applies both with regard to the amounts of CO and H₂ themselves in the gas mixture (G0) and with regard to the relative amounts of CO and H₂ to other constituents of the gas mixture (G0) and especially also with respect to the relative amounts of CO and H₂ based on each other. Thus, there is in principle no restriction whatsoever with respect to the ratio of CO to H₂ in the gas mixture (G0), provided that the gas mixture (G0) allows the conversion of at least some of CO and H₂ in the gas mixture to dimethyl ether and CO₂ in step (3) of the process according to the invention. Thus, for example, the ratio of H₂ in percent by volume to CO in percent by volume may be in the range from 5:95 to 66:34, the H₂ to CO ratio H₂ [% by vol.]: CO [% by vol.] in the gas mixture (G0) being preferably in the range from 10:90 to 66:34, further preferably from 20:80 to 62:38, further preferably from 30:70 to 58:42, further preferably from 40:60 to 55:45, further preferably from 45:55 to 53:47 and further preferably from 48:52 to 52:48. In particularly preferred embodiments of the process according to the invention, the gas mixture (G0) provided in step (1) has a CO to H₂ ratio H₂ [% by vol.]: CO [% by vol.] in the range from 49:51 to 51:49.

With regard to the further constituents of gas mixture (G0) which may optionally be present therein alongside CO and H₂, there is no restriction whatsoever as mentioned above according to the present invention, provided that conversion can be effected to a gas mixture (G1) comprising dimethyl ether and CO₂ in step (3). In particular embodiments of the process according to the invention, the gas mixture (G0) comprises not only CO and H₂ but also CO₂. With regard to the proportion of CO₂ which may be present in the gas mixture (G0) which is provided in step (1), there is correspondingly no restriction whatsoever, the content thereof preferably being within a range which allows achievement of a molar ratio of CO₂ to dimethyl ether in the gas mixture (G1) which, in preferred embodiments of the process according to the invention, is in the range from 10:90 to 90:10 and further preferably in the range from 30:70 to 70:30, further preferably from 40:60 to 60:40, further preferably from 45:55 to 55:45, further preferably from 48:52 to 52:48, further preferably from 49:51 to 51:49, and further preferably from 49.5:50.5 to 50.5:49.5.

In an alternatively preferred embodiment of the process according to the invention, in which CO₂ is present in the gas mixture (G0) in addition to CO and H₂, the gas mixture preferably has a module according to formula (I)

$$\frac{{H}_{2} [ \% by vol . ] - {CO}_{2} [ \% by vol . ]}{CO [ \% by vol . ] + {CO}_{2} [ \% by vol . ]} \qquad ( I )$$

where the module for gas mixture (G0) is in the range from 5:95 to 66:34. In this alternatively preferred embodiment, the module is further preferably in the range from 10:90 to 66:34 and even further preferably in the range from 20:80 to 62:38, further preferably from 30:70 to 58:42, further preferably from 40:60 to 55:45, further preferably from 45:55 to 53:47 and further preferably from 48:52 to 52:48. In a particularly preferred embodiment of the process according to the invention, the module of the formula (I) for gas mixture (G0) is in the range from 49:51 to 51:49.

Alternatively, it is preferred in the process according to the invention that, in the presence of CO₂ in addition to CO and H₂ in gas mixture (G0), the content of CO₂ is within a range which allows a gas mixture (G1) to be obtained, after contacting of gas mixture (G0) with the catalyst (C1) in step (3), in which the CO₂ content is in the range from 20 to 70% by volume based on the total volume of gas mixture (G1) and preferably in the range from 25 to 65% by volume, further preferably from 30 to 60% by volume, further preferably from 35 to 55% by volume, further preferably from 40 to 50% by volume and further preferably from 42 to 48% by volume. In particularly preferred embodiments in which the gas mixture (G0) provided in step (1) comprises CO₂ in addition to CO and H₂, the CO₂ content is within a range which allows, after contacting of gas mixture (G0) with the catalyst (C1), a gas mixture (G1) to be obtained in step (3) which has a CO₂ content in the range from 44 to 46% by volume.

In step (2) of the process according to the invention, a catalyst (C1) is provided for conversion of CO and H₂ to dimethyl ether. With regard to the catalyst (C1), there is no restriction whatsoever, either with regard to the amount in which it can be used or with regard to the composition and nature thereof, provided that it enables the conversion of at least some of the CO and H₂ in the gas mixture (G0) on contacting in step (3) to dimethyl ether and CO₂. In a particularly preferred embodiment of the process according to the invention, catalyst (C1) comprises one or more catalytically active substances for conversion of synthesis gas to methanol and one or more catalytically active substances for dehydration of methanol.

With respect to the one or more catalytically active substances for conversion of synthesis gas to methanol which are present with preference in catalyst (C1), there is no restriction whatsoever in principle, provided that an appropriate conversion of at least some of the CO and H₂ present in gas mixture (G0) to methanol can be effected by contacting gas mixture (G0) with catalyst (C1) in step (3). In a particularly preferred embodiment of the process according to the invention, catalyst (C1) comprises one or more substances selected from the group consisting of copper oxide, aluminum oxide, zinc oxide, ternary oxides and mixtures of two or more thereof as the one or more catalytically active substances for conversion of synthesis gas to methanol. In particularly preferred embodiments thereof, the ternary oxide is a spinel compound, the spinel preferably comprising Zn and/or Al, and the spinel compound further preferably being a Zn—Al spinel.

In a particularly preferred embodiment of the process according to the invention, the one or more substances for conversion of synthesis gas to methanol comprise a mixture of copper oxide, aluminum oxide and zinc oxide. With regard to these particularly preferred embodiments, there are no restrictions in principle with respect to the relative proportions of the individual substances in the mixture, preference being given in accordance with the present invention to mixtures in which copper oxide is present in an amount of 50 to 80% by weight, aluminum oxide in an amount of 2 to 8% by weight and zinc oxide in an amount of 15 to 35% by weight based on the total weight of copper oxide, aluminum oxide and zinc oxide in the catalytically active substance for conversion of synthesis gas to methanol. Further preferably, the mixture comprises copper oxide in an amount of 65 to 75% by weight, aluminum oxide in an amount of 3 to 6% by weight and zinc oxide in an amount of 20 to 30% by weight.

In an alternatively preferred embodiment of the process according to the invention, the one or more substances for conversion of synthesis gas to methanol comprise a mixture of copper oxide, ternary oxide and zinc oxide. With regard to these alternatively preferred embodiments, there are likewise no restrictions in principle with respect to the relative proportions of the individual substances in the mixture, preference being given in accordance with the present invention to mixtures in which copper oxide is present in an amount of 50 to 80% by weight, the ternary oxide in an amount of 15 to 35% by weight and zinc oxide in an amount of 15 to 35% by weight based on the total weight of copper oxide, ternary oxide and zinc oxide in the catalytically active substance for conversion of synthesis gas to methanol. Further preferably, the mixture comprises copper oxide in an amount of 65 to 75% by weight, the ternary oxide in an amount of 20 to 30% by weight and zinc oxide in an amount of 20 to 30% by weight. In particularly preferred embodiments thereof, the ternary oxide is a spinel compound, the spinel preferably comprising Zn and/or Al, and the spinel compound further preferably being a Zn—Al spinel.

With respect to the one or more catalytically active substances for dehydration of methanol which are present with preference in catalyst (C1), there is likewise no restriction whatsoever in principle, provided that the dehydration of the methanol formed from at least some of the CO and H₂ present in gas mixture (G0) can be brought about during the contacting of gas mixture (G0) with catalyst (C1) in step (3). In particularly preferred embodiments of the process according to the invention, the one or more catalytically active substances for dehydration of methanol present with preference in catalyst (C1) preferably comprise one or more compounds selected from the group consisting of aluminum hydroxide, aluminum oxide hydroxide, gamma-aluminum oxide, aluminosilicates, zeolites and mixtures of two or more thereof.

With respect to the aluminosilicates present with preference in the one or more catalytically active substances for dehydration of methanol, these may be selected from the group of the clay minerals, for example the group consisting of kaolin, halloysite, kaolinite, illite, montmorillonite, vermiculite, talc, palygorskite, pyrophyllite and mixtures of two or more thereof.

The zeolites which may be present with preference in the one or more catalytically active substances for dehydration of methanol likewise comprise all zeolites suitable for dehydration of methanol and mixtures thereof, these preferably comprising one or more zeolites selected from the group consisting of zeolite A, zeolite X, zeolite Y, zeolite L, mordenite, ZSM-5, ZSM-11, and mixtures of two or more thereof. In particularly preferred embodiments of the present invention, the one or more catalytically active substances for dehydration comprise one or more zeolites, the one or more zeolites preferably comprising ZSM-5.

With respect to the aluminum oxide hydroxide present with preference in the one or more catalytically active substances for dehydration of methanol, this preferably comprises boehmite.

With regard to the relative amounts of the one or more substances for conversion of synthesis gas to methanol and of the one or more catalytically active substances for dehydration of methanol which may be present in the preferred catalyst (C1), there are no restrictions whatsoever in principle, provided that a gas mixture (G1) comprising dimethyl ether and CO₂ can be obtained in step (3) on contacting of gas mixture (G0) with the catalyst. In particularly preferred embodiments of the process according to the invention, the preferred catalyst (C1) comprises 70-90% by weight of the one or more catalytically active substances for conversion of synthesis gas to methanol and 10-30% by weight of the one or more catalytically active substances for dehydration of methanol, and further preferably 75-85% by weight of the one or more catalytically active substances for conversion of synthesis gas to methanol and 15-25% by weight of the one or more catalytically active substances for dehydration of methanol based on the total weight of the one or more substances for conversion of synthesis gas to methanol and the one or more catalytically active substances for dehydration of methanol.

With respect to the particle size of the one or more catalytically active substances for conversion of synthesis gas to methanol and/or the one or more catalytically active substances for dehydration of methanol, there is likewise no restriction whatsoever in principle, provided that a gas mixture (G1) comprising dimethyl ether and CO₂ can be obtained in step (3) in the contacting of gas mixture (G0) with the catalyst. In particularly preferred embodiments of the process according to the invention. According to the present invention, however, it is preferable that, in the preferred embodiments of catalyst (C1) comprising one or more catalytically active substances for conversion of synthesis gas to methanol and one or more catalytically active substances for dehydration of methanol, these each independently have a particle size D₉₀ in the range from 180 to 800 μm, further preferably in the range from 250 to 800 μm, and further preferably from 350 to 800 μm. In these preferred embodiments, it is further preferable that, in addition to the preferred and particularly preferred particle sizes D₉₀, these have a particle size D₅₀ in the range from 40 to 300 μm, further preferably from 40 to 270 μm, and further preferably from 40 to 220 μm. In these particularly preferred embodiments, it is even further preferable at, in addition to the preferred and particularly preferred particle sizes D₉₀ and D₅₀, the one or more catalytically active substances for conversion of synthesis gas to methanol and the one or more catalytically active substances for dehydration of methanol each independently have a particle size D₁₀ in the range from 5 to 140 μm, further preferably from 5 to 80 μm, and further preferably from 5 to 50 μm.

According to the present invention, the particle size can be determined by any suitable analysis method known to those skilled in the art. In this context, one example would be the use of the Mastersizer 2000 or 3000 measuring instruments from Malvern Instruments GmbH. The particle size D₁₀ corresponds to a diameter at which 10% by weight of the particles examined have a smaller diameter than this. Correspondingly, the particle size D₅₀ indicates a diameter at which 50% by weight of the particles examined have a smaller diameter than this, and the particle size D₉₀, finally, corresponds to the diameter at which 90% by weight of the particles have a smaller diameter.

According to the present invention, catalyst (C1) may comprise one or more substances for enhancing the activity and/or selectivity of the catalyst and especially one or more promoters. In particularly preferred embodiments of catalyst (C1), the one or more catalytically active substances which, in preferred embodiments of the catalyst for dehydration of methanol, are present therein comprise promoters. The promoters present with preference may be present as one or more additional substances in catalyst (C1) or as a dopant in one of the substances present in catalyst (C1), and, in particularly preferred embodiments of catalyst (C1), one or more of the catalytically active substances present therein are doped with one or more promoters. In these particularly preferred embodiments, there is no restriction whatsoever in principle with respect to the catalytically active substances in catalyst (C1) which may be doped with one or more promoters, and so one or more or else all catalytically active substances in catalyst (C1) may be doped with one or more promoters. In particularly preferred embodiments of catalyst (C1), these may be one or more catalytically active substances for conversion of synthesis gas to methanol and/or one or more catalytically active substances for dehydration of methanol, and, in particularly preferred embodiments thereof, the one or more catalytically active substances for dehydration of methanol are doped with one or more promoters. In these particularly preferred embodiments in which one or more of the catalytically active substances for dehydration of methanol are doped with one or more promoters, preference is given to doping aluminum hydroxide and/or aluminum oxide hydroxide and/or gamma-aluminum oxide, and further preference to doping aluminum oxide hydroxide and/or gamma-aluminum oxide, with one or more promoters, the one or more promoters preferably being selected from the group consisting of niobium, tantalum, phosphorus, boron and mixtures of two or more thereof, and further preferably from the group consisting of niobium, tantalum, boron and mixtures of two or more thereof.

Thus, preference is given in accordance with the present invention to embodiments of the process for converting a gas mixture comprising CO and H₂ to olefins in which the catalyst (C1) comprises
one or more catalytically active substances for conversion of synthesis gas to methanol; and
one or more catalytically active substances for dehydration of methanol; the one or more catalytically active substances for conversion of synthesis gas to methanol preferably being selected from the group consisting of copper oxide, aluminum oxide, zinc oxide, ternary oxides and mixtures of two or more thereof.

Further preference is accordingly given in accordance with the present invention to embodiments of the process for converting a gas mixture comprising CO and H₂ to olefins, in which, in the preferred embodiments of catalyst (C1), the one or more catalytically active substances for dehydration of methanol are selected from the group consisting of aluminum hydroxide, aluminum oxide hydroxide, gamma-aluminum oxide, aluminosilicates, zeolites and mixtures of two or more thereof, preference being given to doping of aluminum hydroxide and/or aluminum oxide hydroxide and/or gamma-aluminum oxide with niobium, tantalum, phosphorus and/or boron, further preference to doping with niobium and/or tantalum and/or boron.

With regard to the provision of a gas mixture (G0) comprising CO and H₂ in step (1) of the process according to the invention, there is no restriction whatsoever in principle with respect to the origin thereof and/or with respect to the one or more steps which may precede the provision in step (1) in order to be able to provide a gas mixture (G0) comprising CO and H₂ in the process according to the invention. Thus, the provision of gas mixture (G0) in step (1) may comprise the obtaining of the gas mixture, for example, from any suitable carbon source, the carbon source preferably being selected from the group consisting of oil, coal, natural gas, biomass, carbonaceous wastes and mixtures of two or more thereof. In preferred embodiments of the process according to the invention, the provision of gas mixture (G0) in (1) comprises the obtaining of the gas mixture from a carbon source selected from the group consisting of oil, coal, natural gas, cellulosic materials and/or wastes, landfill waste, agricultural waste and mixtures of two or more thereof, and further preferably from the group consisting of oil, coal, natural gas and mixtures of two or more thereof. In particularly preferred embodiments of the process according to the invention, the provision of gas mixture (G0) in (1) comprises the obtaining of the gas mixture from coal and/or natural gas.

Thus, preference is given to embodiments of the process according to the invention in which the provision of gas mixture (G0) in (1) comprises the obtaining of the gas mixture from a carbon source, the carbon source preferably being selected from the group consisting of oil, coal, natural gas, biomass, carbonaceous wastes and mixtures of two or more thereof. In an alternatively preferred embodiment, the carbon source comprises carbon or hydrocarbon, which means that, in further preferred embodiments, the provision of gas mixture (G0) comprises the conversion of carbon or hydrocarbon to a product comprising hydrogen and carbon monoxide.

In the process according to the invention, in step (3), gas mixture (G0) is contacted with the catalyst (C1) to obtain a gas mixture (G1) comprising dimethyl ether and CO₂. With regard to the conditions for contacting of gas mixture (G0) with catalyst (C1) in step (3), there are no particular restrictions in principle, provided that a gas mixture (G1) comprising dimethyl ether and CO₂ can be obtained. Thus, there are no restrictions whatsoever with respect to the temperature at which the contacting in step (3) is effected, the contacting in (3) in the process according to the invention preferably being effected at a temperature in the range from 150 to 400° C. Further preferably, the contacting in (3) is effected at a temperature in the range from 200 to 350° C., further preferably from 230 to 300° C. and further preferably from 240 to 270° C. In particularly preferred embodiments of the process according to the invention, the contacting in (3) is effected at a temperature in the range from 245 to 255° C.

Thus, preference is given to embodiments of the process according to the invention in which the contacting in (3) is effected at a temperature in the range from 150 to 400° C.

The same applies correspondingly with regard to the pressure at which gas mixture (G0) is contacted with catalyst (C1) in step (3), and so there are initially no restrictions whatsoever in principle here either, provided that a gas mixture (G1) comprising dimethyl ether and CO₂ can be obtained. According to the present invention, however, the contacting in (3) is preferably effected at an elevated pressure relative to the standard pressure of 1.03 kPa. Thus, in these preferred embodiments of the process according to the invention, the contacting in (3) can be effected, for example, at a pressure in the range from 2 to 150 bar, the contacting preferably being effected at a pressure in the range from 5 to 120 bar, further preferably from 10 to 90 bar, further preferably from 30 to 70 bar, further preferably from 40 to 60 bar, further preferably from 45 to 55 bar and further preferably from 47 to 53 bar. In particularly preferred embodiments of the process according to the invention, the contacting in (3) is effected at a pressure in the range from 49 to 51 bar.

Thus, preference is given to embodiments of the process according to the invention in which the contacting in (3) is effected at a pressure in the range from 2 to 150 bar.

In step (4) of the process according to the invention, a catalyst (C2) for conversion of dimethyl ether to olefins is provided. As with respect to catalyst (C1), there is no restriction whatsoever with respect to (C2) either, either with respect to the amount thereof or with respect to the composition and/or nature thereof, provided that it is suitable for converting at least some of the dimethyl ether present in gas mixture (G1) to at least one olefin. According to the present invention, however, a catalyst (C2) comprising one or more zeolites is preferably provided in step (4). With regard to the one or more zeolites preferably present in catalyst (C2), there is again no restriction whatsoever, provided that the conversion of at least some of the dimethyl ether to at least one olefin is possible, preference being given to the presence of zeolites of the MFI, MEL and/or MWW structure type therein.

Thus, preference is given to embodiments of the process according to the invention in which catalyst (C2) comprises one or more zeolites of the MFI, MEL and/or MWW structure type.

If one or more of the zeolites present with preference in catalyst (C2) are of the MWW structure type, there is again no restriction whatsoever with respect to the type and/or number of MWW zeolites which can be used according to the present invention. Thus, these may be selected, for example, from the group of zeolites of the MWW structure type consisting of MCM-22, [Ga—Si—O]-MWW, [Ti—Si—O]-MWW, ERB-1, ITQ-1, PSH-3, SSZ-25 and mixtures of two or more thereof, preference being given to the use of zeolites of the MWW structure type which are suitable for the conversion of dimethyl ether to olefins, especially MCM-22 and/or MCM-36.

The same applies correspondingly to the zeolites of the MEL structure type present with preference in catalyst (C2) in accordance with the present invention, these being selected, for example, from the group consisting of ZSM-11, [Si—B—O]-MEL, boron-D (MFI/MEL mixed crystal), boralite D, SSZ-46, silicalite 2, TS-2 and mixtures of two or more thereof. Here too, preference is given to using those zeolites of the MEL structure type which are suitable for the conversion of dimethyl ether to olefins, especially [Si—B—O]-MEL.

According to the present invention, however, especially zeolites of the MFI structure type are preferably present in catalyst (C2). With regard to these preferred embodiments of the present invention, there is likewise no restriction whatsoever with respect to the type and/or number of the zeolites of this structure type which are used, the one or more zeolites of the MFI structure type present with preference in catalyst (C2) preferably being selected from the group consisting of ZSM-5, ZBM-10, [As—Si—O]-MFI, [Fe—Si—O]-MFI, [Ga—Si—O]-MFI, AMS-1B, AZ-1, boron-C, boralite C, encilite, FZ-1, LZ-105, monoclinic H-ZSM-S, mutinaite, NU-4, NU-5, silicalite, TS-1, TSZ, TSZ-III, TZ-01, USC-4, USI-108, ZBH, ZKQ-1B, ZMQ-TB and mixtures of two or more thereof. Further preferably, according to the present invention, the catalyst comprises ZSM-5 and/or ZBM-10 as the zeolite of the MFI structure type, particular preference being given to using ZSM-5 as the zeolite. With regard to the zeolitic material ZBM-10 and the preparation thereof, reference is made, for example, to EP 0 007 081 A1 and to EP 0 034 727 A2, the content of which, particularly with regard to the preparation and characterization of the material, is hereby incorporated into the present invention.

Thus, preference is given in accordance with the present invention to embodiments of catalyst (C2) in which the one or more zeolites are of the MFI structure type.

In a preferred embodiment of the present invention, catalyst (C2) does not comprise any significant amounts of one or more nonzeolitic materials and especially does not comprise any significant amounts of one or more aluminophosphates (AlPOs or APOs) or of one or more aluminosilicophosphates (SAPOs). In the context of the present invention, catalyst (C2) is essentially free of or does not comprise any significant amounts of a specific material in cases in which this specific material is present in the catalyst in an amount of 1% by weight or less in the catalyst, based on the total weight of catalyst (C2) and preferably based on 100% by weight of the total amount of the one or more zeolites of the MFI, MEL and/or MWW structure type present with preference in catalyst (C2), and preferably comprises it in an amount of 0.5% by weight or less, further preferably of 0.1% by weight or less, further preferably of 0.05% by weight or less, further preferably of 0.001% by weight or less, further preferably of 0.0005% by weight or less and further preferably in an amount of 0.0001% by weight or less. A specific material in the context of the present invention particularly denotes a particular element or a particular combination of elements, a particular substance or a particular substance mixture, and also combinations and/or mixtures of two or more thereof.

The aluminophosphates (AlPOs and APOs) in the context of the present invention generally include all crystalline aluminophosphate phases. The aluminosilicophosphates (SAPOs) in the context of the present invention generally include all crystalline aluminosilicophosphate phases and especially the SAPO materials SAPO-11, SAPO-47, SAPO-40, SAPO-43, SAPO-5, SAPO-31, SAPO-34, SAPO-37, SAPO-35, SAPO-42, SAPO-56, SAPO-18, SAPO-41, SAPO-39 and CFSAPO-1A.

In particularly preferred embodiments of the process according to the invention in which catalyst (C2) comprises one or more zeolites, and especially one or more zeolites of the MFI, MEL and/or MWW structure type, the one or more zeolites comprise one or more alkaline earth metals. In general, according to the present invention, there is no restriction whatsoever either with regard to the type and/or the number of alkaline earth metals present with preference in the one or more zeolites, or with regard to the manner in which they may be present in the one or more zeolites. Thus, the one or more zeolites may comprise one or more alkaline earth metals selected, for example, from the group consisting of magnesium, calcium, strontium, barium and combinations of two or more thereof. According to the present invention, the one or more alkaline earth metals, however, are preferably selected from the group consisting of magnesium, calcium, strontium and combinations of two or more thereof, and, in particularly preferred embodiments of the inventive catalyst, the alkaline earth metal is magnesium. In alternatively preferred embodiments of the present invention, the catalyst does not comprise any, or any significant amounts of, calcium and/or strontium.

Thus, according to the present invention, preference is given to embodiments of catalyst (C2) in which the in the one or more zeolites of the MFI, MEL and/or MWW structure type comprise one or more alkaline earth metals, the one or more alkaline earth metals preferably being selected from the group consisting of Mg, Ca, Sr, Ba and combinations of two or more thereof, further preferably consisting of Mg, Ca, Sr and combinations of two or more thereof, the alkaline earth metal more preferably being Mg.

With regard to the manner in which the one or more alkaline earth metals are present in the one or more zeolites in the preferred catalyst (C2), these may in principle be present in the micropores of the one or more zeolites and/or as a constituent of the zeolitic skeleton, especially at least partly in isomorphic substitution for an element in the zeolite skeleton, preferably for silicon and/or aluminum as a constituent of the zeolite skeleton and more preferably at least partly in isomorphic substitution for aluminum. With regard to the presence of the one or more alkaline earth metals in the micropores of the one or more zeolites, these may be present as a separate compound, for example as a salt and/or oxide therein, and/or as a positive counterion to the zeolite skeleton. According to the present invention, the one or more alkaline earth metals are present at least partly in the pores and preferably in the micropores of the one or more zeolites, and, further preferably, the one or more alkaline earth metals are present therein at least partly as the counterion of the zeolite skeleton, as can arise, for example, in the course of production of the one or more zeolites in the presence of the one or more alkaline earth metals and/or can be brought about by performance of an ion exchange with the one or more alkaline earth metals in the zeolite already produced.

With regard to the amount of the one or more alkaline earth metals which may be present in the particularly preferred embodiments of catalyst (C2), as already noted above, there are no particular restrictions according to the present invention with regard to the amount in which they may be present in the one or more zeolites. It is thus possible in principle for any possible amount of the one or more alkaline earth metals to be present in the one or more zeolites, for example in a total amount of the one or more alkaline earth metals of 0.1-20% by weight based on the total amount of the one or more zeolites. According to the present invention, however, it is preferred that the one or more alkaline earth metals are present in a total amount in the range of 0.5-15% by weight based on 100% by weight of the total amount of the one or more zeolites, further preferably of 1-10% by weight, further preferably of 2-7% by weight, further preferably of 3-5% by weight and further preferably of 3.5-4.5% by weight. In particularly preferred embodiments of the present invention, the one or more alkaline earth metals are present in a total amount of 3.8-4.2% by weight in the one or more zeolites. For all of the above percentages by weight for alkaline earth metal in the one or more zeolites, these are calculated proceeding from the one or more alkaline earth metals as the metal.

Thus, further preference is given in accordance with the present invention to embodiments of catalyst (C2) for the conversion of dimethyl ether to olefins in which the one or more preferred zeolites are of the MFI, MEL and/or MWW structure type which comprise the one or more alkaline earth metals in a total amount in the range from 0.1 to 20% by weight, based in each case on the total amount of the one or more zeolites of the MFI, MEL and/or MWW structure type and calculated as the metal.

In further preferred embodiments of the process according to the invention, catalyst (C2) comprises, as well as the above-described zeolites according to the particular and preferred embodiments as described in the application, further particles of one or more metal oxides. According to the present invention, there are no restrictions whatsoever either with respect to the type of metal oxides which may preferably be used in catalyst (C2), or with respect to the number of different metal oxides which may be present therein. According to the present invention, however, preference is given to metal oxides which are generally used in catalytic materials as inert materials and especially as support substances, preferably with a large BET surface area. According to the present invention, figures for surface areas of a material are preferably based on the BET (Brunauer-Emmett-Teller) surface area thereof, this preferably being determined to DIN 66131 by nitrogen absorption at 77 K.

With regard to the metal oxides which may preferably be present in catalyst (C2), there are no restrictions whatsoever. It is thus possible in principle to use any suitable metal oxide compound and mixtures of two or more metal oxide compounds. Preference is given to using metal oxides which are thermally stable in processes for the conversion of dimethyl ether to olefins, the metal oxides preferably serving as binders. Thus, the one or more metal oxides which are used with preference in catalyst (C2) are preferably selected from the group consisting of alumina, titania, zirconia, aluminum-titanium mixed oxides, aluminum-zirconium mixed oxides, aluminum-lanthanum mixed oxides, aluminum-zirconium-lanthanum mixed oxides, titanium-zirconium mixed oxides and mixtures of two or more thereof. Further preferably, according to the present invention, the one or more metal oxides are selected from the group consisting of alumina, aluminum-titanium mixed oxides, aluminum-zirconium mixed oxides, aluminum-lanthanum mixed oxides, aluminum-zirconium-lanthanum mixed oxides, and mixtures of two or more thereof. According to the present invention, particular preference is given to using the metal oxide alumina as particles in the catalyst. According to the present invention, it is further preferred that the metal oxide present with preference in catalyst (C2) is at least partly in amorphous form.

In even further preferred embodiments of the process according to the invention, the particles of the one or more metal oxides which, in the particular and preferred embodiment as described in the present application, are present in catalyst (C2) comprise phosphorus. With respect to the form in which the phosphorus is present in the particles of the one or more metal oxides, according to the present invention, there is no particular restriction whatsoever, provided that at least some of the phosphorus is in oxidic form. According to the present invention, phosphorus is in oxidic form if it is in present in conjunction with oxygen, i.e. if at least some of the phosphorus is at least partly in a compound with oxygen, especially with covalent bonding of at least some of the phosphorus to the oxygen. According to the present invention, it is preferred that the phosphorus which is at least partly in oxidic form comprises oxides of phosphorus and/or oxide derivatives of phosphorus. The oxides of phosphorus according to the present invention include especially phosphorus trioxide, diphosphorus tetroxide, phosphorus pentoxide and mixtures of two or more thereof. In addition, according to the present invention, it is preferred that the phosphorus and especially the phosphorus in oxidic form is at least partly in amorphous form, the phosphorus and especially the phosphorus in oxidic form further preferably being present essentially in amorphous form. According to the present invention, the phosphorus and especially the phosphorus in oxidic form is essentially in amorphous form when the proportion of phosphorus and especially of phosphorus in oxidic form which is present in crystalline form in the catalyst is 1% by weight or less based on 100% by weight of the total amount of the particles of the one or more metal oxides, the phosphorus being calculated as the element, preferably in an amount of 0.5% by weight or less, further preferably of 0.1% by weight or less, further preferably of 0.05% by weight or less, further preferably of 0.001% by weight or less, further preferably of 0.0005% by weight or less and further preferably in an amount of 0.0001% by weight or less.

With regard to the manner in which the phosphorus which is at least partly in oxidic form is present in the one or more metal oxides of the preferred embodiments of catalyst (C2), according to the present invention, there is no particular restriction whatsoever, either with respect to the manner in which it is present or with respect to the amount of phosphorus present in the one or more metal oxides. With respect to the manner in which the phosphorus is present, it may thus in principle be applied to the one or more metal oxides as the element and/or as one or more independent compounds and/or incorporated in the one or more metal oxides, for example in the form of a dopant of the one or more metal oxides, this especially comprising embodiments in which the phosphorus and the one or more metal oxides at least partly form mixed oxides and/or solid solutions. According to the present invention, the phosphorus is preferably applied partly in the form of one or more oxides and/or oxide derivatives to the one or more metal oxides in the particles, the one or more oxides and/or oxide derivatives of phosphorus further preferably originating from a treatment of the one or more metal oxides with one or more acids of phosphorus and/or with one or more of the salts thereof. The one or more acids of phosphorus preferably refer to one or more acids selected from the group consisting of phosphinic acid, phosphonic acid, phosphoric acid, peroxophosphoric acid, hypodiphosphonic acid, diphosphonic acid, hypodiphosphoric acid, diphosphoric acid, peroxodiphosphoric acid and mixtures of two or more thereof. Further preferably, the one or more phosphoric acids are selected from the group consisting of phosphonic acid, phosphoric acid, diphosphonic acid, diphosphoric acid and mixtures of two or more thereof, further preferably from the group consisting of phosphoric acid, diphosphoric acid and mixtures thereof, and, in particularly preferred embodiments of the present invention, the phosphorus present with preference in the one or more metal oxides at least partly originates from a treatment of the one or more metal oxides with phosphoric acid and/or with one or more phosphate salts.

In further embodiments which are particularly preferred in accordance with the present invention, the one or more zeolites of the MFI, MEL and/or MWW structure type present with preference in catalyst (C2) likewise comprise phosphorus. With regard to the form in which the phosphorus is present in the one or more zeolites, the same applies as described in the present application with respect to phosphorus present in the one or more metal oxides likewise present with preference in catalyst (C2), especially with regard to the partial presence thereof in oxidic form. With respect to the manner in which the phosphorus may be present in the one or more zeolites, according to the present invention, it is preferably present in the pores of the zeolite skeleton and especially in the micropores thereof, either as an independent phosphorus-comprising compound and/or as a counterion to the zeolite skeleton, the phosphorus more preferably being present at least partly as an independent compound in the pores of the zeolite skeleton.

Thus, according to the present invention, particular preference is given to embodiments of catalyst (C2) for the conversion of dimethyl ether to olefins in which the one or more zeolites of the MFI, MEL and/or MWW structure type present with preference in catalyst (C2) comprise phosphorus, the phosphorus being at least partly in oxidic form.

With regard to the ratio in which the one or more zeolites of the MFI, MEL and/or MWW structure type on the one hand and the particles of one or more metal oxides on the other hand are present in catalyst (C2) in particularly preferred embodiments of the process according to the invention, there is no particular restriction in principle. Thus, the weight ratio of zeolite to metal oxide in the catalyst according to the particular and preferred embodiments of the present invention may, for example, be in the range from 10:90 to 95:5. According to the present invention, the zeolite:metal oxide weight ratio, however, is preferably in the range from 20:80 to 90:10, further preferably in the range from 40:60 to 80:20 and further preferably in the range from 50:50 to 70:30. In particularly preferred embodiments of the present invention, the zeolite:metal oxide weight ratio is in the range from 55:45 to 65:45. In the context of the present invention, the zeolite:metal oxide weight ratio indicates especially the weight ratio of the total weight of the one or all of the plurality of the zeolites to the total weight of the particles of the one or all of the plurality of metal oxides.

Thus, in the preferred embodiments process according to the invention, preference is given to using embodiments of catalyst (C2) in which the weight ratio of zeolite:metal oxide in the catalyst is in the range from 10:90 to 95:5.

With regard to the amount of phosphorus present in the embodiments of catalyst (C2) which are used with particular preference in the process according to the invention, there is no restriction whatsoever in principle, and so any conceivably possible contents of phosphorus may be present in catalyst (C2). Thus, the total amount of phosphorus in catalyst (C2) according to the present invention may, for example, be in the range of 0.1-20% by weight, the total amount of phosphorus being based on the sum of the total weight of zeolites of the MFI, MEL and/or MWW structure type and the total weight of the particles of the one or more metal oxides, the phosphorus being calculated as the element. In particularly preferred embodiments of the catalyst (C2) used, the total amount of phosphorus in the catalyst, however, is preferably in the range of 0.5-15% by weight, further preferably in the range of 1-10% by weight, further preferably of 2-7% by weight, further preferably of 2.5-5% by weight, further preferably of 3.5-4.5% by weight, further preferably of 3.3-4.2% by weight and further preferably of 3.5-4% by weight. In particularly preferred embodiments of the present invention, the total amount of phosphorus in the catalyst (C2) used with particular preference, based on the sum of the total weight of zeolites and the total weight of the particles of the one or more metal oxides, is in the range of 3.7-3.9% by weight, the phosphorus being calculated as the element.

Thus, in particularly preferred embodiments of the process according to the invention, preference is given to using embodiments of catalyst (C2) in which the total amount of phosphorus, based on the sum of the total weight of zeolites of the MFI, MEL and/or MWW structure type and the total weight of the particles of the one or more metal oxides and calculated as the element, is in the range from 0.1 to 20% by weight. In the process according to the invention, particular preference is thus given to embodiments in which catalyst (C2) comprises one or more zeolites of the MFI, MEL and/or MWW structure type and particles of one or more metal oxides, the one or more zeolites preferably being of the MFI structure type. Preference is further given to embodiments in which the one or more zeolites of the MFI, MEL and/or MWW structure type comprise one or more alkaline earth metals, preferably Mg. Irrespective of this, further preference is also given to embodiments thereof in which the one or more zeolites of the MFI, MEL and/or MWW structure type comprise phosphorus and/or the particles of the one or more metal oxides comprise phosphorus, the phosphorus being present in each case at least partly in oxidic form.

With regard to the form in which catalyst (C2) is used in the process according to the invention, there are likewise no restrictions whatsoever, and so, in the particularly preferred embodiments of the catalyst (C2) used, the one or more zeolites and the particles of the one or more metal oxides present therein may in principle be combined in any possible and suitable manner to form a catalyst. In preferred embodiments of the process according to the invention, catalyst (C2) is provided in step (4) in the form of a shaped body, and, in the particularly preferred embodiments of the catalyst (C2) used, the shaped body comprises a mixture of the one or more zeolites of the MFI, MEL and/or MWW structure type and the particles of the one or more metal oxides, preferably of the one or more zeolites and the particles of the one or more metal oxides according to one of the particular or preferred embodiments as described in the present application. In a further preferred embodiment of the present invention, catalyst (C2) is provided in step (4) in the form of an extrudate.

In step (5) of the process according to the invention, the gas mixture (G1) comprising dimethyl ether and optionally CO₂ is contacted with catalyst (C2) to obtain an olefin-comprising gas mixture (G2). With regard to the content of dimethyl ether and any CO₂ present in gas mixture (G1), there is no restriction whatsoever in principle, provided that some of the dimethyl ether can be converted in step (5) to at least one olefin. This applies in principle both with respect to the absolute amounts of dimethyl ether and any CO₂ which may be present in gas mixture (G1) and with regard to the relative amounts of dimethyl ether and any CO₂ based on any further constituents of gas mixture (G1), and also with regard to the ratio thereof with respect to one another. With regard to the absolute amount of CO₂ which may be present in gas mixture (G1), gas mixture (G1) may, for example, have a CO₂ content in the range from 20 to 70% by volume based on the total volume of the gas mixture. In preferred embodiments of the process according to the invention, gas mixture (G1) preferably has a CO₂ content in the range from 25 to 65% by volume based on the total volume of the gas mixture, and further preferably from 30 to 60% by volume, further preferably from 35 to 55% by volume, further preferably from 40 to 50% by volume and further preferably from 42 to 48% by volume. In particularly preferred embodiments of the process according to the invention, gas mixture (G1) has a CO₂ content in the range from 44 to 46% by volume based on the total volume of the gas mixture.

Preference is thus given to embodiments of the process according to the invention in which the gas mixture (G1) which is contacted with (C2) in (5) has a CO₂ content in the range from 20 to 70% by volume based on the total volume of the gas mixture.

According to the present invention, the composition of gas mixture (G1) as per the particular and preferred embodiments defined herein is based either on the composition of the gas mixture which is obtained in step (3) after the contacting with the catalyst (C1) or on the composition of the gas mixture (G1) which is contacted with catalyst (C2) in (5), or else on the composition of gas mixture (G1) between steps (3) and (5). Thus, the composition of gas mixture (G1) as obtained in step (3) immediately after the contacting may differ from the composition of gas mixture (G1) immediately prior to the contacting in step (5) with catalyst (C2), especially if one or more intermediate steps are effected between steps (3) and (5) in which gas mixture (G1) is treated in a manner which leads, either through removal of at least some of one or more of the components thereof and/or supply of one or more gas streams to gas mixture (G1), to a change in the composition thereof. In preferred embodiments of the process according to the invention, particularly H₂O, methanol, CO and/or H₂ are fully or partly removed, and/or CO₂ is not removed or is fully or partly removed. The term “removal” according to the present invention especially comprehends the controlled removal of a particular component, such that an inevitable loss of CO₂ and/or dimethyl ether in the case of a selective removal, which is possible in principle, of H₂O, CO and/or H₂ between steps (3) and (5) is preferably not considered as a removal of CO₂ and/or dimethyl ether in the context of the present invention.

Thus, preference is given to embodiments of the process according to the invention in which CO₂ present in gas mixture (G1) is removed fully or partly between steps (3) and (5). Particular preference is given to embodiments of the process according to the invention in which only CO₂ is removed fully or partly from gas mixture (G1) between steps (3) and (5). If CO₂ is removed between steps (3) and (5), a CO₂ stream which has the comparatively high pressure present after step (3) is obtained. This is advantageous since the CO₂ removed can be recycled into the synthesis gas production with only slight compression, if any. In the case of recycling of CO₂ after step (5), in contrast, a pressure increase is absolutely necessary owing to the pressure drop which has occurred. In the case of partial removal of the CO₂ between steps (3) and (5), a further advantage is found to be that the proportion of CO₂ can be adjusted as desired in step (5). For a partial removal, it is especially advisable to use a membrane having the appropriate characteristics.

In particularly preferred embodiments of the process according to the invention, however, no components are removed from gas mixture (G1) between steps (3) and (5) and/or no further gas streams are supplied, and, further preferably, there is neither removal of components from gas mixture (G1) nor supply of further gas streams, and so the composition of gas mixture (G1) immediately after the contacting in step (3) is the same as the composition of the same gas mixture (G1) immediately prior to the contacting thereof with catalyst (C2) in step (5).

Thus, preference is given to embodiments of the process according to the invention in which no CO₂ is removed from gas mixture (G1) between steps (3) and (5). Particular preference is given to embodiments of the process according to the invention in which no components are removed from gas mixture (G1) nor are any further gas streams supplied thereto between steps (3) and (5).

With regard to the content of dimethyl ether in gas mixture (G1), there are accordingly no restrictions in principle either, provided that at least some of the dimethyl ether can be converted to at least one olefin in the contacting of gas mixture (G1) with catalyst (C2) in step (5). Thus, gas mixture (G1) may have a content of dimethyl ether which is, for example, in the range from 20 to 70% by volume based on the total volume of the gas mixture. According to the present invention, however, preference is given to embodiments of the process for converting a gas mixture comprising CO and H₂ to olefins in which the content of dimethyl ether in gas mixture (G1) is in the range from 25 to 65% by volume and further preferably from 30 to 60% by volume, further preferably from 35 to 55% by volume, further preferably from 40 to 50% by volume and further preferably from 42 to 48% by volume. In particularly preferred embodiments of the process according to the invention, gas mixture (G1) has a content of dimethyl ether in the range from 44 to 46% by volume based on the total volume of the gas mixture.

Thus, preference is given in accordance with the present invention to embodiments of the process for converting a gas mixture comprising CO and H₂ to olefins in which gas mixture (G1) which is contacted with (C2) in (5) has a content of dimethyl ether in the range from 20 to 70% by volume based on the total volume of the gas mixture.

Regardless of the absolute amounts of CO₂ and dimethyl ether present in gas mixture (G1), preference is given to embodiments of the process according to the invention in which gas mixture (G1) has a molar ratio of CO₂ to dimethyl ether in the range from 10:90 to 90:10. Preference is additionally given to molar ratios of CO₂ to dimethyl ether in gas mixture (G1) in the range from 30:70 to 70:30 and further preferably from 40:60 to 60:40, further preferably from 45:55 to 55:45, further preferably from 48:52 to 52:48 and further preferably from 49:51 to 51:49. In particularly preferred embodiments of the process according to the invention, gas mixture (G1) has a molar ratio of CO₂ to dimethyl ether in the range from 49.5:50.5 to 50.5:49.5.

Thus, preference is given in accordance with the present invention to embodiments of the process for converting a gas mixture comprising CO and H₂ to olefins in which, in (5), the molar CO₂: dimethyl ether ratio of gas mixture (G1) which is contacted with (C2) in (5) is in the range from 10:90 to 90:10.

As well as any CO₂ and dimethyl ether, further substances may also be present in gas mixture (G1) in the contacting operation in step (5), especially those substances which were present in gas mixture (G0), and especially CO and/or H₂ which have not been converted fully to dimethyl ether and CO₂ in step (2), and also substances which have formed in addition to dimethyl ether and CO₂ on contacting of gas mixture (G0) with catalyst (C1) in step (3). Thus, gas mixture (G1) in the contacting operation in step (5) may comprise not only dimethyl ether and any CO₂ but also H₂. In the embodiments of the process according to the invention in which gas mixture (G1) has an H₂ content, there is no restriction whatsoever in principle with regard to the amounts in which H₂ may be present therein, provided that they allow the contacting of gas mixture (G1) with catalyst (C2) in step (5) for conversion of at least some of the dimethyl ether to at least one olefin.

Thus, gas mixture (G1) may have an H₂ content of, for example, up to 35% by volume based on the total volume of the gas mixture. In the present process, gas mixture (G1), however, preferably has an H₂ content in the range from 0.1 to 30% by volume based on the total volume of the gas mixture, gas mixture (G1) further preferably having an H₂ content of 0.5 to 25% by volume, further preferably from 1 to 22% by volume, further preferably from 2 to 20% by volume, further preferably from 3 to 18% by volume, further preferably from 4 to 15% by volume and further preferably from 4.5 to 12% by volume. In particularly preferred embodiments of the process according to the invention, gas mixture (G1) has an H₂ content in the range from 5 to 10% by volume based on the total volume of the gas mixture.

Thus, further preference is given in accordance with the present invention to embodiments of the process for converting a gas mixture comprising CO and H₂ to olefins in which gas mixture (G1) which is contacted with (C2) in (5) has an H₂ content in the range from 0 to 35% by volume based on the total volume of the gas mixture.

Regardless of the absolute amounts of H₂ and dimethyl ether present in gas mixture (G1), preference is given to embodiments of the process according to the invention in which gas mixture (G1) has a molar ratio of H₂ to dimethyl ether in the range from 0 to 64:36. Preference is additionally given to molar ratios of H₂ to dimethyl ether in gas mixture (G1) in the range from 0.2:99.8 to 55:45 and further preferably from 1:99 to 45:55, further preferably from 4:96 to 36:64, further preferably from 7:93 to 27:73 and further preferably from 9:91 to 22:78. In particularly preferred embodiments of the process according to the invention, gas mixture (G1) has a molar ratio of H₂ to dimethyl ether in the range from 10:90 to 19:81.

Thus, preference is given in accordance with the present invention to embodiments of the process for converting a gas mixture comprising CO and H₂ to olefins in which, in (5), the molar H₂:dimethyl ether ratio of gas mixture (G1) which is contacted with (C2) in (5) is in the range from 0 to 64:36.

As already mentioned, as well as CO₂ and dimethyl ether and possibly CO and/or H₂, further substances which may also be present in gas mixture (G1) are those which, alongside those substances which were in gas mixture (G0), have not been formed until step (2) as an intermediate and/or by-product and, in the case of the intermediates, have not been converted fully to dimethyl ether and CO₂, and particular mention should be made here of methanol. Thus, gas mixture (G1) may comprise not only dimethyl ether and CO₂ and possibly CO and/or H₂ but also methanol. In the embodiments of the process according to the invention in which gas mixture (G1) has a methanol content, there is no restriction whatsoever in principle with regard to the amounts in which methanol may be present therein, provided that they allow the contacting of gas mixture (G1) with catalyst (C2) in step (5) for conversion of at least some of the dimethyl ether to at least one olefin.

Thus, gas mixture (G1) may have a methanol content of, for example, up to 20% by volume based on the total volume of the gas mixture. In the present process, gas mixture (G1), however, preferably has a methanol content in the range from 0.1 to 15% by volume based on the total volume of the gas mixture, gas mixture (G1) further preferably having a methanol content of 0.5 to 14% by volume, further preferably from 1 to 13% by volume, further preferably from 1.5 to 12% by volume, further preferably from 2 to 11% by volume, further preferably from 3 to 10% by volume and further preferably from 4 to 9% by volume. In particularly preferred embodiments of the process according to the invention, gas mixture (G1) has a methanol content in the range from 5 to 8% by volume based on the total volume of the gas mixture.

Thus, further preference is given in accordance with the present invention to embodiments of the process for converting a gas mixture comprising CO and H₂ to olefins in which gas mixture (G1) which is contacted with (C2) in (5) has a content of methanol in the range from 0 to 20% by volume based on the total volume of the gas mixture.

Regardless of the absolute amounts of methanol and dimethyl ether present in gas mixture (G1), preference is given to embodiments of the process according to the invention in which gas mixture (G1) has a molar ratio of methanol to dimethyl ether in the range from 0.1:99.9 to 50:50. Preference is additionally given to molar ratios of methanol to dimethyl ether in gas mixture (G1) in the range from 0.5:99.5 to 30:70 and further preferably from 1:99 to 20:80, further preferably from 2:98 to 15:85, further preferably from 3:97 to 13:87 and further preferably from 4:96 to 10:90. In particularly preferred embodiments of the process according to the invention, gas mixture (G1) has a molar ratio of methanol to dimethyl ether in the range from 5:95 to 7:93.

Thus, preference is given in accordance with the present invention to embodiments of the process for converting a gas mixture comprising CO or H₂ to olefins in which, in (5), the molar methanol:dimethyl ether ratio of gas mixture (G1) which is contacted with (C2) in (5) is in the range from 0.1:99.9 to 50:50.

With regard to the substances which may be present alongside CO₂ and dimethyl ether and possibly CO and/or H₂ and/or methanol in gas mixture (G1), these may also comprise H₂O, and these substances may already be present in gas mixture (G0) and/or form in the course of contacting of gas mixture (G0) with catalyst (C1) in step (3) as a by-product and/or intermediate owing to incomplete conversion of gas mixture (G0) to dimethyl ether and CO₂. Thus, gas mixture (G1) in the contacting operation in step (5) may comprise not only dimethyl ether and any CO₂ but also H₂O. In the embodiments of the process according to the invention in which gas mixture (G1) has an H₂O content, there is no restriction whatsoever in principle with regard to the amounts in which H₂O may be present therein, provided that they allow the contacting of gas mixture (G1) with catalyst (C2) in step (5) for conversion of at least some of the dimethyl ether to at least one olefin.

Thus, gas mixture (G1) in the contacting operation in step (5) may have an H₂O content of, for example, up to 20% by volume based on the total volume of the gas mixture. In the present process, gas mixture (G1), however, preferably has an H₂O content in the range from 0.1 to 15% by volume based on the total volume of the gas mixture, gas mixture (G1) further preferably having an H₂O content of 0.5 to 14% by volume, further preferably from 1 to 13% by volume, further preferably from 1.5 to 12% by volume, further preferably from 2 to 11% by volume, further preferably from 3 to 10% by volume and further preferably from 4 to 9% by volume. In particularly preferred embodiments of the process according to the invention, gas mixture (G1) has an H₂O content in the range from 5 to 8% by volume based on the total volume of the gas mixture.

Thus, further preference is given in accordance with the present invention to embodiments of the process for converting a gas mixture comprising CO and H₂ to olefins in which gas mixture (G1) which is contacted with (C2) in (5) has an H₂O content in the range from 0 to 20% by volume based on the total volume of the gas mixture.

Regardless of the absolute amounts of H₂O and dimethyl ether present in gas mixture (G1), preference is given to embodiments of the process according to the invention in which gas mixture (G1) has a molar ratio of H₂O to dimethyl ether in the range from 0 to 22:78. Preference is additionally given to molar ratios of H₂O to dimethyl ether in gas mixture (G1) in the range from 0.5:99.5 to 20:80 and further preferably from 1:99 to 19:81, further preferably from 3:97 to 18:82, further preferably from 6:94 to 17:83 and further preferably from 8:92 to 16:84. In particularly preferred embodiments of the process according to the invention, gas mixture (G1) has a molar ratio of H₂O to dimethyl ether of 10:90 to 15:85.

Thus, preference is given in accordance with the present invention to embodiments of the process for converting a gas mixture comprising CO and H₂ to olefins in which, in (5), the molar H₂O: dimethyl ether ratio of gas mixture (G1) which is contacted with (C2) in (5) is in the range from 0 to 22:78.

In the process according to the invention, in step (5), gas mixture (G1) is contacted with the catalyst (C2) to obtain an olefin-comprising gas mixture (G2). With regard to the conditions for contacting of gas mixture (G1) with catalyst (C2) in step (5), there are no particular restrictions in principle, provided that a gas mixture (G2) comprising at least one olefin can be obtained. Thus, there are no restrictions whatsoever with respect to the temperature at which the contacting in step (5) is effected, the contacting in (5) in the process according to the invention preferably being effected at a temperature in the range from 150 to 800° C. The contacting in (5) is further preferably effected at a temperature in the range from 200 to 750° C., further preferably from 250 to 700° C., further preferably from 300 to 650° C., further preferably from 350 to 600° C., further preferably from 400 to 580° C. and further preferably from 430 to 560° C. In particularly preferred embodiments of the process according to the invention, the contacting in (5) is effected at a temperature in the range from 450 to 500° C.

Thus, preference is given in accordance with the present invention to embodiments of the process for converting a gas mixture comprising CO and H₂ to olefins in which the contacting in (5) is effected at a temperature in the range from 150 to 800° C.

The same applies correspondingly with regard to the pressure at which gas mixture (G1) is contacted with catalyst (C2) in step (5), and so there initially are no restrictions whatsoever in principle here either, provided that a gas mixture (G2) comprising at least one olefin can be obtained. Thus, in the process according to the invention, the contacting in (5) can be effected, for example, at a pressure in the range from 0.1 to 20 bar, the contacting preferably being effected at a pressure in the range from 0.3 to 10 bar, further preferably from 0.5 to 5 bar, further preferably from 0.7 to 3 bar, further preferably from 0.8 to 2.5 bar and further preferably from 0.9 to 2.2 bar. In particularly preferred embodiments of the process according to the invention, the contacting in (5) is effected at a pressure in the range from 1 to 2 bar.

Thus, preference is given in accordance with the present invention to embodiments of the process for converting a gas mixture comprising CO and H₂ to olefins in which the contacting in (5) is effected at a pressure in the range from 0.1 to 20 bar.

In addition, there are no particular restrictions with respect to the manner of performance of the process according to the invention for converting a gas mixture comprising CO and H₂ to olefins, and so it is possible to use either a continuous or a noncontinuous process, the noncontinuous process being performable, for example, as a batch process. According to the present invention, however, it is preferable to conduct at least some of the process according to the invention for converting a gas mixture comprising CO and H₂ to olefins as a continuous process. Thus, preference is given in accordance with the present invention to embodiments of the process for converting a gas mixture comprising CO and H₂ to olefins in which at least part of the process is performed continuously.

With respect to these preferred embodiments of an at least partially continuous process, there are no restrictions whatsoever with respect to the space velocities selected in the continuous process regime, provided that the conversion of a gas mixture comprising CO and H₂ to at least one olefin can be effected. In particular embodiments of the process according to the invention in which step (3) is conducted in continuous mode, it is possible, for example, to select space velocities in the contacting in step (3) in the range from 50 to 50 000 h⁻¹, preference being given to selecting a space velocity from 100 to 20 000 h⁻¹, further preferably from 500 to 15 000 h⁻¹, further preferably from 1000 to 10 000 h⁻¹, further preferably from 1500 to 7500 h⁻¹, further preferably from 2000 to 5000 h⁻¹, further preferably from 2200 to 2700 h⁻¹ and further preferably from 2300 to 2500 h⁻¹. In particularly preferred embodiments of the process according to the invention for converting a gas mixture comprising CO and H₂ to olefins, space velocities for the contacting of gas mixture (G0) with catalyst (C1) in step (3) in the range from 2350 to 2450 h⁻¹ are selected.

Thus, preference is given in accordance with the present invention to embodiments of the process for converting a gas mixture comprising CO and H₂ to olefins in which the space velocity in the contacting in (3) in the range from 50 to 50 000 h⁻¹.

With regard to particularly preferred embodiments of the process according to the invention in which the contacting of the gas mixture (G1) comprising dimethyl ether and optionally CO₂ with catalyst (C2) in (5) is conducted in continuous mode, it is possible, for example, to select space velocities in the range from 0.3 to 50 h⁻¹, preference being given to selecting space velocities in the range from 0.5 to 40 h⁻¹, further preferably from 1 to 30 h⁻¹, further preferably from 1.5 to 20 h⁻¹, further preferably from 2 to 15 h⁻¹ and further preferably from 2.5 to 10 h⁻¹. In particularly preferred embodiments of the process according to the invention for converting a gas mixture comprising CO and H₂ to olefins, space velocities for the contacting of gas mixture (G1) with catalyst (C2) in step (5) in the range from 3 to 5 h⁻¹ are selected.

Thus, preference is given in accordance with the present invention to embodiments of the process for converting a gas mixture comprising CO and H₂ to olefins in which the space velocity in the contacting in (5) are in the range from 0.3 to 50 h⁻¹.

According to the present invention, the term “space velocity” refers to the loading of the catalyst calculated as grams of dimethyl ether per gram of catalyst per hour based on the contacting of gas mixture (G1) with catalyst (C2) in step (5), or to the loading of the catalyst in grams of methanol per gram of catalyst per hour based on the contacting of gas mixture (G0) with catalyst (C1) in step (3).

The present invention comprises the following embodiments, these especially also comprising the specific combinations of the individual embodiments which are defined by the corresponding dependency references:
1. A process for converting a gas mixture comprising CO and H₂ to olefins, comprising
   (1) providing a gas mixture (G0) comprising CO and H₂;
   (2) providing a catalyst (C1) for conversion of CO and H₂ to dimethyl ether;
   (3) contacting the gas mixture (G0) with the catalyst (C1) to obtain a gas mixture (G1) comprising dimethyl ether and CO₂;
   (4) providing a catalyst (C2) for conversion of dimethyl ether to olefins;
   (5) contacting the gas mixture (G1) comprising dimethyl ether with the catalyst (C2) to obtain an olefin-comprising gas mixture (G2).
2. The process according to embodiment 1, wherein the CO₂ present in gas mixture (G1) is fully or partly removed between steps (3) and (5). Therefore, it is thus possible to remove other components as well as CO₂.
3. The process according to embodiment 2, wherein only CO₂ is fully or partly removed from gas mixture (G1) between steps (3) and (5).
4. The process according to embodiment 1, wherein no CO₂ is removed from gas mixture (G1) between steps (3) and (5).
5. The process according to embodiment 4, wherein no components are removed from gas mixture (G1) nor are any further gas streams supplied thereto between steps (3) and (5).
6. The process according to any of embodiments 1 to 5, wherein the gas mixture (G1) which is contacted with (C2) in (5) has a CO₂ content in the range from 20 to 70% by volume based on the total volume of the gas mixture.
7. The process according to any of embodiments 1 to 6, wherein the module according to formula (I):

$$\frac{{H}_{2} [ \% by vol . ] - {CO}_{2} [ \% by vol . ]}{CO [ \% by vol . ] + {CO}_{2} [ \% by vol . ]} \qquad ( I )$$

for the gas mixture (G0) is in the range from 5:95 to 66:34.
8. The process according to any of embodiments 1 to 7, wherein the gas mixture (G1) which is contacted with (C2) in (5) has a content of dimethyl ether in the range from 20 to 70% by volume based on the total volume of the gas mixture.
9. The process according to any of embodiments 1 to 8, wherein the molar CO₂:dimethyl ether ratio in (5) of the gas mixture (G1) which is contacted with (C2) in (5) is in the range from 10:90 to 90:10.
10. The process according to any of embodiments 1 to 9, wherein the gas mixture (G1) which is contacted with (C2) in (5) has an H₂ content in the range from 0 to 35% by volume based on the total volume of the gas mixture.
11. The process according to any of embodiments 1 to 10, wherein the molar H_{2:}dimethyl ether ratio in (5) of the gas mixture (G1) which is contacted with (C2) in (5) is in the range from 0 to 64:36.
12. The process according to any of embodiments 1 to 11, wherein the gas mixture (G1) which is contacted with (C2) in (5) has a content of methanol in the range from 0 to 20% by volume based on the total volume of the gas mixture.
13. The process according to any of embodiments 1 to 12, wherein the molar methanol:dimethyl ether ratio in (5) of the gas mixture (G1) which is contacted with (C2) in (5) is in the range from 0.1:99.9 to 50:50.
14. The process according to any of embodiments 1 to 13, wherein the gas mixture (G1) which is contacted with (C2) in (5) has an H₂O content in the range from 0 to 20% by volume based on the total volume of the gas mixture.
15. The process according to any of embodiments 1 to 14, wherein the molar H₂O:dimethyl ether ratio in (5) of the gas mixture (G1) which is contacted with (C2) in (5) is in the range from 0 to 22:78.
16. The process according to any of embodiments 1 to 15, wherein the provision of gas mixture (G0) in (1) comprises the obtaining of the gas mixture from a carbon source.
17. The process according to embodiment 16, wherein the provision of gas mixture (G0) comprises the conversion of carbon or hydrocarbon to a product comprising hydrogen and carbon monoxide.
18. The process according to any of embodiments 1 to 17, wherein the contacting in (3) is effected at a temperature in the range from 150 to 400° C., preferably from 200 to 300° C.
19. The process according to any of embodiments 1 to 18, wherein the contacting in (3) is effected at a pressure in the range from 2 to 150 bar, preferably from 20 to 70 bar, more preferably from 30 to 50 bar.
20. The process according to any of embodiments 1 to 19, wherein the contacting in (5) is effected at a temperature in the range from 150 to 800° C.
21. The process according to any of embodiments 1 to 20, wherein the contacting in (5) is effected at a pressure in the range from 0.1 to 20 bar.
22. The process according to any of embodiments 1 to 21, wherein at least part of the process is performed continuously.
23. The process according to embodiment 22, wherein the space velocity in the contacting in (3) is in the range from 50 to 50 000 h⁻¹.
24. The process according to embodiment 22 or 23, in which the space velocity in the contacting in (5) is in the range from 0.3 to 50 h⁻¹, preferably in the range from 0.5 to 40 h⁻¹, further preferably from 1 to 30 h⁻¹.
25. The process according to any of embodiments 1 to 24, wherein catalyst (C1) comprises
   one or more catalytically active substances for conversion of synthesis gas to methanol; and
   one or more catalytically active substances for dehydration of methanol.
26. The process according to embodiment 25, wherein the one or more catalytically active substances for conversion of synthesis gas to methanol are selected from the group consisting of copper oxide, aluminum oxide, zinc oxide, ternary oxides and mixtures of two or more thereof.
27. The process according to embodiment 25 or 26, wherein the one or more catalytically active substances for dehydration of methanol are selected from the group consisting of aluminum hydroxide, aluminum oxide hydroxide, gamma-aluminum oxide, aluminosilicates, zeolites and mixtures of two or more thereof.
28. The process according to any of embodiments 25 to 27, wherein the one or more catalytically active substances for dehydration of methanol are doped with niobium, tantalum, phosphorus and/or boron.
29. The process according to any of embodiments 1 to 28, wherein catalyst (C2) comprises one or more zeolites of the MFI, MEL and/or MWW structure type and particles of one or more metal oxides, the one or more zeolites preferably being of the MFI structure type.
30. The process according to embodiment 29, wherein the one or more zeolites of the MFI, MEL and/or MWW structure type comprise one or more alkaline earth metals, preferably Mg.
31. The process according to embodiment 29 or 30, wherein the one or more zeolites of the MFI, MEL and/or MWW structure type comprise phosphorus, the phosphorus being present at least partly in oxidic form.
32. The process according to any of embodiments 29 to 31, wherein the particles of the one or more metal oxides comprise phosphorus, the phosphorus being present at least partly in oxidic form.
33. A process for preparing olefins from carbon or hydrocarbon, comprising:
   a first synthesis step (21) wherein carbon or hydrocarbon is converted to a first product (11) comprising hydrogen and carbon monoxide,
   a second synthesis step (23) wherein hydrogen and carbon monoxide are converted to a second product (13) comprising dimethyl ether and carbon dioxide,
   a third synthesis step (25) for preparation of olefins wherein dimethyl ether is converted to a third product (15) comprising olefins (especially ethylene and propylene),
      which comprises
   feeding the second product (13) directly to the third synthesis step (25), or merely removing CO₂ from the second product (13) and then supplying the second product (14) to the third synthesis step (25).
34. The process according to embodiment 33, wherein the first synthesis step (21) and second synthesis step (23) are performed at essentially equal pressures. More particularly, the pressure at the outlet of the first synthesis step (21) differs from the pressure at the inlet of the second synthesis step (23) by less than 3 bar, preferably by less than 1 bar.
35. The process according to embodiment 33 or 34, wherein methane is reacted in the first synthesis step (21) with water or oxygen to give hydrogen and carbon monoxide.
36. The process according to embodiment 33 or 34, wherein the first synthesis step (21) is a dry reforming step which converts methane and carbon dioxide to hydrogen and carbon monoxide.
37. The process according to any of embodiments 33 to 36, wherein the first synthesis step (21) and the second synthesis step (23) are performed at a pressure in the range from 20 bar to 70 bar, preferably from 30 bar to 50 bar.
38. The process according to any of embodiments 33 to 37, wherein carbon monoxide and hydrogen are converted in the second synthesis step (23) to dimethyl ether and carbon dioxide until a juncture from which dimethyl ether is present in a concentration of at least 60%, 70%, 80%, 90% or 100% of the equilibrium concentration of dimethyl ether.
39. The process according to any of embodiments 33 to 38, wherein carbon dioxide is separated from the second product (13) in a first separation step (24).
40. The process according to embodiment 39, wherein the carbon dioxide removed in the first separation step (24) is used for preparation (21) of synthesis gas.
41. The process according to any of embodiments 33 to 40, wherein a predominantly hydrogen-, carbon monoxide- and methane-containing residual gas (18) is removed from the third product (15) in a second separation step (26).
42. The process according to embodiment 41, wherein the predominantly hydrogen-, carbon monoxide- and methane-containing residual gas (18) removed is supplied to the first synthesis step (21).
43. The process according to embodiment 41, wherein the predominantly hydrogen-, carbon monoxide- and methane-containing residual gas (18) is used for provision of thermal energy for the first synthesis step (21).
44. The process according to embodiments 33 to 43, wherein heat which arises in the second synthesis step (23) and/or the third synthesis step (25) is used to generate energy.
45. The process according to embodiment 44, wherein heat which arises in the second synthesis step (23) and/or the third synthesis step (25) is used to drive turbines, especially in the second separation step (26).

The present invention thus also comprises a process comprising a first synthesis step wherein carbon or hydrocarbon is converted to a first product (synthesis gas) comprising hydrogen and carbon monoxide, a second synthesis step wherein hydrogen and carbon monoxide are converted to a second product comprising dimethyl ether and carbon dioxide, and a third synthesis step wherein dimethyl ether is converted to a third product comprising olefins (especially ethylene and propylene).

Accordingly, the second product (DME) is supplied without further treatment, except for the optional removal of carbon dioxide from the second product, to the third synthesis step (olefin preparation).

Synthesis gas can be prepared in the first synthesis step by coal gasification from carbon and water or oxygen. Alternatively, synthesis gas can be prepared by autothermal reforming, steam reforming or partial oxidation of hydrocarbons. Preference is given to preparing synthesis gas in the first synthesis step from methane, particular preference to preparing it by steam reforming, partial oxidation or dry reforming.

The second synthesis step in the context of the invention is understood to mean the direct dimethyl ether synthesis, in which dimethyl ether is formed directly from hydrogen and carbon monoxide.

The third synthesis step, the olefin synthesis, can be performed in the presence of suitable catalysts, for example zeolite or silicon-aluminum-phosphate catalysts.

“Without further treatment” in the context of the invention means that - apart from an optional carbon dioxide removal - the product of the second synthesis step is supplied directly to the third synthesis step, the olefin synthesis, without any change in the composition or purification.

In one embodiment of the invention, the first synthesis step and the second synthesis step are performed at essentially equal pressures, preferably at equal pressures. Essentially equal pressures within the understanding of the invention are pressures which differ from one another by not more than 1 bar, preferably 0.5 bar, more preferably 0.4 bar, 0.3 bar, 0.2 bar, and most preferably not more than 0.1 bar. An equal pressure in the context of this configuration is understood to mean that the pressure between the two synthesis steps does not differ by any more than the extent caused by the normal pressure drop of the components required in between.

In a further embodiment of the invention, methane is reacted with water or oxygen to give hydrogen and carbon monoxide in the first synthesis step. Methane in the context of the invention also comprises methane-containing gases such as natural gas.

In a preferred embodiment of the invention, the first synthesis step is a dry forming step wherein methane and carbon dioxide are converted to hydrogen and carbon monoxide.

Dry reforming in the context of the invention is understood to mean the conversion of methane or natural gas and CO₂ with supply of heat and in the absence of water to synthesis gas having a stoichiometric ratio of H₂ and CO of about 1:1. Dry reforming in the context of the invention also comprises the conversion of CI⁻14 or natural gas and CO₂ in the presence of water vapor, water being present only in a stoichiometric ratio to methane or natural gas of 1:2, 1:3, 1:4, 1:5, 1:10 or 1:20.

Generally, in the context of this invention, reference is made to dry reforming when the molar ratio of water to carbon in the feed is less than 2:1, preferably less than 1:1.

The dry reforming and/or the direct dimethyl ether synthesis can be performed in the presence of suitable catalysts, for instance transition metal catalysts. In dry reforming, modified soot-resistant Ni-based catalysts are especially advantageous, as also used in other steam reforming processes. In the dimethyl ether synthesis, it is advantageous to use copper-based catalysts which are also commonly used in other methanol synthesis processes.

In a further preferred embodiment of the invention, the process is performed at a pressure of 20 bar to 50 bar. An increase in the pressure can shift the equilibrium of the reaction to the product side and thus increase the yield of the reaction.

In a further preferred embodiment of the invention, in the second synthesis step, carbon monoxide and hydrogen are converted to dimethyl ether and carbon dioxide up to a juncture from which dimethyl ether is present in a concentration of at least 60%, 70%, 80%, 90% or 100% of the equilibrium concentration of dimethyl ether.

The equilibrium concentration of dimethyl ether in the context of the invention means the dimethyl ether concentration which is present when the reaction of carbon monoxide and hydrogen to give dimethyl ether and carbon dioxide is at chemical equilibrium. The chemical equilibrium of the reaction has been attained when the rate of the forward reaction (3 H₂+3 CO→DME+CO₂) is equal to the rate of the reverse reaction (DME+CO₂→3 H₂+3 CO).

In a further embodiment of the invention, in a first separation step, carbon dioxide is removed from the second product. Carbon dioxide can be removed from the second product by conventional separation processes, for example distillation, for example by amine or alkali metal carbonate washes, washes with organic solvents such as methanol, N-methyl-2-pyrrolidone or polyethylene glycol dimethyl ether, or using a membrane.

In a further embodiment of the invention, the carbon dioxide removed in the first separation step is used for preparation of synthesis gas, wherein carbon dioxide and methane are converted to hydrogen and carbon monoxide.

In a further embodiment, in a second separation step, a predominantly hydrogen-, carbon monoxide- and methane-containing residual gas is removed from the third product, forming a fourth product comprising olefins (especially ethylene and propylene).

In a further embodiment of the invention, the predominantly hydrogen-, carbon monoxide- and methane-containing residual gas removed is supplied to the first synthesis step or the second synthesis step, in which case it is possible to convert methane in the first synthesis step to synthesis gas and hydrogen, and carbon monoxide in the second synthesis step to dimethyl ether and carbon dioxide. This reuse of the residual gas increases the yield of the process and reduces the amount of waste products.

In an alternative embodiment of the invention, the predominantly hydrogen-, carbon monoxide-and methane-containing residual gas is used for provision of thermal energy for synthesis gas preparation, especially for steam reforming or dry reforming. Thermal energy can be generated by oxidation of the combustible constituents of the residual gas to water and carbon dioxide. Supply of thermal energy or heat to the endothermic reforming step can shift the chemical equilibrium of the reforming reaction to the product side (hydrogen and carbon monoxide).

In a further embodiment of the invention, the heat which arises in the second and/or third synthesis step is used to generate energy.

In a further embodiment of the invention, the heat which arises in the second synthesis step and/or the third synthesis step is used in the form of steam to drive turbines, especially in the second separation step. The use of the heat which arises increases the economic viability of the process.

A basic idea of the present invention consists, more particularly, in an integration of the three process steps of synthesis gas preparation 21, direct DME synthesis 23 and olefin synthesis 25.

In a preferred embodiment (FIG. 1), synthesis gas 11 is prepared 21 from carbon or hydrocarbon, preferably from methane. According to the method used, the synthesis gas 11 formed may have a stoichiometric ratio of hydrogen to carbon monoxide of greater than 1:1 (e.g. 3:1). The ratio of hydrogen and carbon monoxide of 1:1 needed for the direct DME synthesis 23 can be achieved by removal of the excess hydrogen 22. If the synthesis gas 11 is prepared by dry reforming 21, there is no hydrogen removal 22. The synthesis gas 11, 12 may also comprise unconverted reactants of the synthesis gas preparation 21, such as methane and carbon dioxide. Subsequently, the synthesis gas 11, 12 is used in the direct DME synthesis 23. The product 13 of the DME synthesis 23 may optionally be freed 24, 14 from carbon dioxide still present, or it is supplied directly to the olefin synthesis 25 without further treatment. The product of the olefin synthesis 15 can in turn optionally be freed 24 of carbon dioxide and is subsequently subjected to a separation 26 from olefin 17 and predominantly hydrogen-, carbon monoxide- and methane-containing residual gas 18. The residual gas 18 can in turn be supplied to the synthesis gas preparation 21.

A further preferred embodiment comprises the following features:
synthesis gas provision 21 and DME synthesis 23 are performed at the same pressure level (in the range of 30-50 bar) (- no compressor needed upstream of DME stage 23),
no purification/workup of the synthesis gas upstream of the DME stage 23 (apart from any H₂ removal 22),
the DME direct synthesis is brought close to the chemical equilibrium within this pressure range (preferably at 25 bar to 35 bar),
product gas 13 from the DME direct synthesis 23 is conducted without further treatment (CO₂ removal 24 at most) into the DMTO (dimethylether-to-olefin) stage 25,
the waste heat from the DME stage 23 and the DMTO stage 25 is combined and utilized for turbines (preferably in the DMTO separation sequence 26),
the residual gas 18 from the DMTO stage 25 (H₂/CO/CH4) is recycled physically or in the form of energy to the synthesis gas preparation 21,
in the case of dry reforming 21 as the synthesis gas stage 21, the CO₂ formed in the DME step 23 is recycled physically in the dry reforming 21.

The preferred embodiments described above offer especially the following advantages:
streamlining of the process
omission of the compressor stage between synthesis gas preparation 21 and DME synthesis 23,
omission of complex purification steps for the synthesis gas 11, 12 and the DME (particularly through high conversion in the DME stage),
improved thermal integration through use of the waste heat from DME stage 23 and DMTO stage 25 for the energy-intensive separation 26 of the olefin products.

In the case of restriction to dry reforming 21 as the synthesis gas technology, the H₂ removal 22 from the synthesis gas 11 is dispensed with, since the dry reforming 21 forms synthesis gas 11 in a stoichiometric ratio of 1:1.

### DESCRIPTION OF THE FIGURES

FIG. 1 shows a block diagram of a process according to the invention, wherein the reference numerals represent the following:

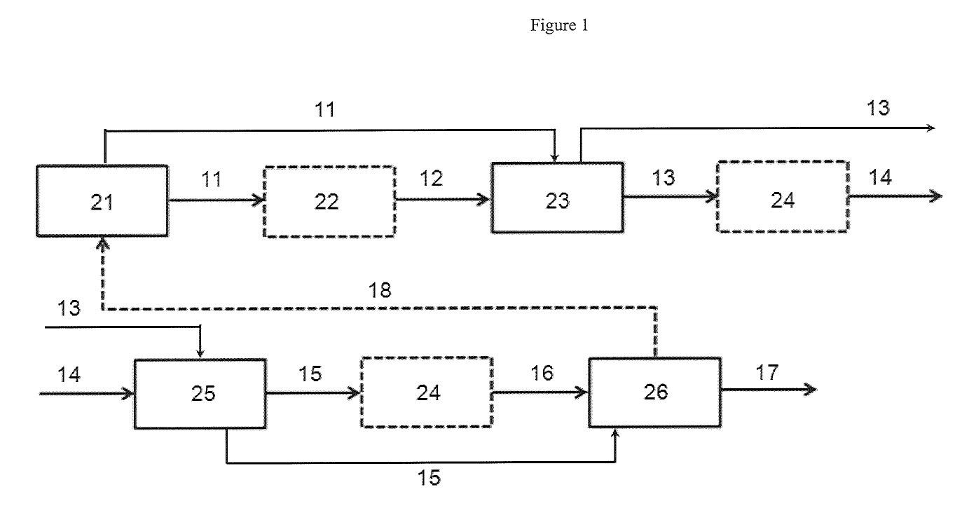

“11” first product (predominantly H₂, CO),

“12” first product after removal of excess H₂ (predominantly H₂ and CO),

“13” second product (predominantly DME, CO₂ with H₂, CO, MeOH, H₂O),

“14” second product after CO₂ removal (predominantly DME, with H₂, CO, MeOH, H₂O),

“15” third product (predominantly olefin),

“16” third product after CO₂ removal,

“17” third product (only olefin),

“18” residual gas (H₂, CO, CH₄),

“21” synthesis gas preparation,

“22” H₂ removal,

“23” direct DME synthesis,

“24” CO₂ removal,

“25” olefin synthesis,

and “26” separation of olefin and residual gas.

### EXAMPLES

### Reference Example 1

### Preparation of a Catalyst for Conversion of Synthesis Gas to Methanol

1.33 kg of copper nitrate, 2.1 kg of zinc nitrate and 0.278 kg of aluminum nitrate were dissolved in 15 l of water in order to obtain a first solution 1. Separately from this, 2.344 kg of sodium hydrogencarbonate were dissolved in 15 l of water in order to obtain a second solution 2. The two solutions were each heated to 90° C., and solution 1 was added rapidly to solution 2 within 1-2 min while stirring. The resulting solution was stirred for a further 15 min and the precipitate formed was then filtered off and washed with distilled water until it was free of nitrates. The filtercake was dried at 110° C. and then dried under a nitrogen atmosphere at 270° C. for 4 h. The metal content of the catalyst in mol% was: Cu=38.8, Zn=48.8 and Al=12.9.

### Reference Example 2

### Preparation of a Catalyst for Conversion of Synthesis Gas to Methanol

2.66 kg of copper nitrate, 1.05 kg of zinc nitrate and 0.278 kg of aluminum nitrate were dissolved in 15 l of water in order to obtain a first solution 1. Separately from this, 2.344 kg of sodium hydrogencarbonate are dissolved in 15 l of water in order to obtain a second solution 2. The two solutions were combined as described in reference example 1 and the precipitate formed was filtered off correspondingly. The metal content in the catalyst according to reference example 2 calculated in mol% was: Cu=61.6, Zn=28.1 and Al=10.9.

### Reference Example 3

### Preparation of a Catalyst for Conversion of Synthesis Gas to Methanol

An aqueous solution of sodium hydrogencarbonate (20%) was prepared by dissolving sodium hydrogencarbonate in 44 kg of distilled water. In addition, a Zn/Al solution was prepared, consisting of 6.88 kg of zinc nitrate and 5.67 kg of aluminum nitrate, and also 23.04 kg of water. The two solutions were heated to 70° C. A vessel filled with 12.1 l of distilled water was likewise heated to 70° C. The solutions prepared were added simultaneously to the initial charge of water, and the addition was effected in such a way that the pH of 7 was maintained during the addition until all of the Zn/Al solution had been added. Subsequently, the resulting mixture having a pH of 7 was stirred for 15 h. The resulting suspension was filtered and washed with distilled water until the wash water had a sodium oxide content of <0.10% and was essentially free of nitrates. The filtercake was dried at 120° C. for 24 h and then calcined under an air stream at 350° C. for 1 h.

### Reference Example 4

### Preparation of a Catalyst for Conversion of Synthesis Gas to Methanol

An aqueous sodium hydrogencarbonate solution (20%) was prepared by dissolving 25 kg of sodium bicarbonate in 100 kg of distilled water. A Cu/Zn nitrate solution was likewise prepared, consisting of 26.87 kg of copper nitrate and 5.43 kg of zinc nitrate, and also 39 kg of water. The two solutions were heated to 70° C. Once the Cu/Zn nitrate solution had reached a temperature of 70° C., the product from the first precipitation was added gradually and the pH was set to a pH=2 by addition of 65% nitric acid. A vessel containing 40.8 l of distilled water was likewise heated to 70° C. The sodium hydrogencarbonate solution and the Cu/Zn nitrate solution were added simultaneously to the initial charge of distilled water, and the addition was effected in such a way that a pH=6.7 was maintained until complete addition of the Cu/Zn nitrate solution. The resulting mixture was subsequently stirred for 10 h, in the course of which the pH, if necessary, was kept at a pH of pH 6.7 by addition of the 65% nitric acid. The resulting suspension was subsequently filtered and washed with distilled water until the wash water had a sodium oxide content <0.10% and was essentially free of nitrate. The filtercake was dried at 120° C. for 72 h and then calcined under an air stream at 300° C. for 3 h. The resulting catalyst consisted of 70% by weight of CuO, 5.5% by weight of Al₂O₃ and 24.5% by weight of ZnO.

### Reference Example 5

### Preparation of a Catalyst for Conversion of Synthesis Gas to dimethyl ether and CO₂

The catalytically active substance for conversion of synthesis gas to methanol from reference example 4 (“Me30” hereinafter) and ZSM-5 as a catalytically active substance for dehydration of methanol were compacted separately in a press for production of tablets and/or in a device for production of pellets. Samples of the catalyst were used in each case with the ZSM-5 zeolites “ZSM5-400H” (elemental analysis: Al=0.238 g/100 g; Na=0.09 g/100 g; Si=45.5 g/100 g; Si:Al (molar)=190.0), “ZSM5-100H” (elemental analysis: Al=0.84 g/100 g; Na=0.02 g/100 g; Si=44 g/100 g; Si:Al (molar)=50.3), “ZSM5-80H” (elemental analysis: Al=0.99 g/100 g; Na<0.01 g/100 g; Si=44 g/100 g; Si:Al (molar)=42.7), “ZSM5-50H” (elemental analysis: Al=1.7 g/100 g; Na=0.02 g/100 g; Si=43 g/100 g; Si:Al (molar)=24.3), and “ZSM5-25H” (elemental analysis: Al=2.7 g/100 g; Na=0,06 g/100 g; Si=41 g/100 g; Si:Al (molar)=14.6) in each case.

The shaped body obtained in each case (diameter=approx. 25 mm; height=approx. 2 mm) was pushed through sieves of a suitable mesh size to obtain the desired spall fraction. The desired amounts of the two fractions were weighed in (9/1, 8/2 or 7/3 of catalytically active substance for conversion of synthesis gas to methanol/catalytically active substance for dehydration of methanol) and then blended with the further components (Heidolph Reax 2 or Reax 20/12) in a mixer.

Reference example 6: Process for conversion of synthesis gas to dimethyl ether and CO₂ 5 ccm of a sample of the catalyst from reference example 5 were installed into a tubular reactor (internal diameter=0.4 cm, embedded in a metal heating element) on a catalyst bed support consisting of alumina powder as a material for the inert layer and then reduced at standard pressure with a mixture of 1% by volume of H₂ and 99% by volume of N₂. In the course of this, the temperature was increased at intervals of 8 h from 150° C. to 170° C. and from 170° C. to 190° C. and finally to 230° C. The synthesis gas mixture consisted of 45% by volume of H₂ and 45% by volume of CO and 10% by volume of inert gas (argon). The catalytically active body was run at an inlet temperature of 250° C. and a gas hourly space velocity (GHSV) of 2400 h⁻¹ and a pressure of 50 bar.

An experiment with a pelletized material according to reference example 5 was also tested, in which the production of pellets (size: 3×3 mm) was not followed by subsequent further processing to spall. Under similar conditions to those for the unpelletized materials, the process was conducted using the same steps. In contrast, however, a tubular reactor having an internal diameter of 3 cm rather than an internal diameter of 0.4 cm was used. Accordingly, the experiments with the pelletized materials were performed at a catalyst volume of 100 ccm.

The results of the experiments are shown in table 1. In table 1, all gas streams were analyzed by online gas chromatography. Argon gas was used as the internal standard for correlation of the incoming and outgoing gas streams. In the experiments, the catalyst for conversion of synthesis gas to methanol “Me30” and ZSM-5 with different Al, Na and Si ratios were used in each case in an Me30: ZSM-5 weight ratio of 8:2. The different mixtures of the spall fractions (corresponding D₁₀, D₅₀ and D₉₀ values for Me30 and ZSM5-100H are shown in table 2) show different CO conversions. With regard to the selectivities, it can be inferred from the results in table 1 that, within the samples in which mainly dimethyl ether is formed, a comparable selectivity for dimethyl ether and CO₂ can be observed. This shows that all catalysts have a sufficient water/gas shift activity, which is required in order to allow the water which forms in the dehydration of methanol to react with CO in order to obtain CO₂ and H₂. Apart from in experiment 4, all catalysts also have a high activity with respect to the dehydration of methanol.

TABLE 1
CO conversion and selectivities for methanol, dimethyl ether, CO₂ and for by-products
in experiments 1 to 10.

| | Molar | | | | | | |
|---|---|---|---|---|---|---|---|
| | Si:Al in | Spall | | Selectivity | | | |
| Exp. | ZSM5 | fraction | C(CO)⁽¹⁾ | MeOH⁽²⁾ | DME⁽³⁾ | CO₂⁽⁴⁾ | Remainder^{(6),(5)} |
| 1 | 14.6 | 0.05-0.1 | 38.5% | 2.46 | 48.45 | 48.7 | 0.39 |
| 2 | 24.3 | 0.05-0.1 | 70.59% | 1.89 | 48.39 | 49.37 | 0.35 |
| 3 | 50.3 | 0.05-0.1 | 73.46% | 1.06 | 49.09 | 49.23 | 0.63 |
| 4 | 190.0 | 0.05-0.1 | 15.92% | 96.2 | 0.44 | 1.33 | 2.03 |
| 5 | 50.3 | 0.05-0.1 | 73.46% | 1.06 | 49.09 | 49.23 | 0.63 |
| 6 | 50.3 | 0.1-0.15 | 65.86% | 2.86 | 50.95 | 46.12 | 0.07 |
| 7 | 50.3 | 0.15-0.2 | 81.43% | 2.91 | 50.22 | 46.79 | 0.08 |
| 8 | 50.3 | 0.2-0.5 | 79.43% | 1.91 | 51.88 | 48.17 | 0.08 |
| 9 | 50.3 | 0.5-0.7 | 61.88% | 3.76 | 48.69 | 47.67 | 0.07 |
| 10 | 50.3 | (pellet) | 80.78% | 1.73 | 49.17 | 48.89 | 0.21 |

⁽¹⁾The CO conversion is calculated as follows: (COᵢₙ − (COₒᵤₜ × argonᵢₙ/argonₒᵤₜ))/COᵢₙ × 100%
⁽²⁾S(MeOH) = volume (MeOH) in product stream/volume (MeOH + dimethyl ether + CO₂ + residual constituents except hydrogen and CO) in product stream × 100%
⁽³⁾S(DME) = volume of dimethyl ether in product stream/volume (MeOH + dimethyl ether + CO₂ + residual constituents except hydrogen and CO) in product stream × 100%
⁽⁴⁾S(CO₂) = volume of CO₂ in product stream/volume (MeOH + dimethyl ether + CO₂ + residual constituents except hydrogen and CO) in product stream × 100%
⁽⁵⁾S(remainder) = volume of the residual constituents in product stream/volume (MeOH + dimethyl ether + CO₂ + residual constituents except hydrogen and CO) in product stream × 100%
⁽⁶⁾“Remainder” is compounds which are formed by the reaction of hydrogen and CO in the reactor except for methanol, dimethyl ether or CO₂.

TABLE 2
D₁₀, D₅₀ and D₉₀ values of the spall fractions of Me30 and ZSM5-100H.

| Component | Spall fraction | D₁₀ [μm] | D₅₀ [μm] | D₉₀ [μm] |
|---|---|---|---|---|
| Me30 | 0.05-0.1 | 2.42 | 46.57 | 89.14 |
| Me30 | 0.1-0.15 | 5.06 | 129.53 | 143.06 |
| Me30 | 0.15-0.2 | 6.33 | 131.69 | 189.23 |
| Me30 | 0.2-0.5 | 20.71 | 275.6 | 396.86 |
| ZSM5-100H | 0.05-0.1 | 2.87 | 56.38 | 82.17 |
| ZSM5-100H | 0.1-0.15 | 5.47 | 100.92 | 184.78 |
| ZSM5-100H | 0.15-0.2 | 5.27 | 163.57 | 196.22 |
| ZSM5-100H | 0.2-0.5 | 5.15 | 373.09 | 489.57 |

### Reference Example 7

### Preparation of a phosphorus-Containing Catalyst for Conversion of a Gas Stream Comprising dimethyl ether and CO₂ to olefins

H-ZSM-5 powder (SiO₂/Al₂O₃=100, ZEO-cat PZ2-100 H from Zeochem) was spray-impregnated with a dilute phosphorus solution. This spray impregnation involved spraying to 90% of the water absorption in order to avoid an excessively wet product. The amount of phosphorus weighed in was such that the powder after the calcination consists of 4% by weight of phosphorus. For impregnation, 400 g of zeolite powder were introduced into a round-bottom flask and installed into a rotary evaporator. 62 g of 85% phosphoric acid were made up to 216 ml of total liquid with distilled water, corresponding to the water absorption. Then the dilute phosphoric acid solution was introduced into a dropping funnel, and sprayed gradually onto the powder (with rotation) via a glass spray nozzle (flooded with 100 l/h of N₂). Subsequently, the powder was dried in a vacuum drying cabinet at 80° C. for 8 h, calcined under air at 500° C. (heating time 4 h), ground to a small size with the aid of an analytical mill and sieved through a 1 mm sieve. The elemental analysis of the product gave a phosphorus content of 3.2-3.3 g/100 g.

The P-ZSM-5 powder thus produced was processed further with Pural SB (Sasol) as a binder to give extrudates, such that the zeolite/binder ratio in the calcined product is 60:40. For this purpose, 380 g of P-ZSM-5 and 329 g of Pural SB were weighed in, mixed and etched with formic acid, Walocel was added thereto and the mixture was processed with 350 ml of water to give a homogeneous material. The kneaded material was forced with the aid of an extrudate press through a 2.5 mm die at approx. 110-115 bar. Subsequently, these extrudates were dried in a drying cabinet at 120° C. for 16 h, calcined under air in a muffle furnace at 500° C. (heating time 4 h) for 4 h and processed in a sieving machine with 2 steel balls (diameter approx. 2 cm, 258 g/ball) to give 1.6-2 mm spall.

The spall thus produced is impregnated with phosphorus in a further step. Prior to the impregnation, the water absorption capacity of the extrudate was determined (3 ml of H₂O/5 g of extrudate). Accordingly, a solution of 74 g of 85% phosphoric acid was made up to 292 ml of total liquid with distilled water. The amount of phosphoric acid was calculated such that, after the calcination, 4% by weight of phosphorus is present on the extrudate. 486 g of spall were initially charged in a spray impregnation drum. The dilute phosphoric acid was sprayed gradually onto the spall (with rotation) via a glass spray nozzle (flooded with 100 l/h of air). The drying was effected at 80° C. in a vacuum drying cabinet for 8 h and the calcination at 500° C. in a muffle furnace under air (heating time 4 h) for 4 h. The elemental analysis of the product gave a phosphorus content of 5.6 g/100 g.

### Reference Example 8

### Preparation of a Magnesium-Containing Catalyst for Conversion of a Gas Stream Comprising dimethyl ether and CO₂ to olefins

H-ZSM-5 powder (SiO2/Al₂O₃=100, ZEO-cat PZ2-100 H from Zeochem) was spray-impregnated with a magnesium nitrate solution. The amount of Mg weighed in was such that the powder after the calcination consists of 4% by weight of magnesium. For impregnation, 58.7 g of zeolite powder were introduced into a round-bottom flask and installed into a rotary evaporator. 43.9 g of magnesium nitrate were brought into solution in water while heating, and made up to 54 ml of total liquid with distilled water, corresponding to the water absorption. Then the dilute magnesium nitrate solution was introduced into a dropping funnel, and sprayed gradually onto the powder (with rotation) via a glass spray nozzle (flooded with 100 l/h of N₂). In the intervening period, the flask is removed and the flask is shaken by hand in order to achieve homogeneous distribution. After approx. 10 min of further rotation time, the powder was dried in a quartz rotary sphere flask at 120° C. for 16 h, calcined under 20 l/h of air at 500° C. (heating time 4 h) for 4 h, ground to a small size with the aid of an analytical mill and sieved through a 1 mm sieve. The elemental analysis of the product gave a magnesium content of 3.7 g/100 g.

The Mg-ZSM-5 powder thus produced was processed further with Pural SB as a binder to give extrudates, such that the zeolite/binder ratio in the calcined product is again 60:40. For this purpose, 58.7 g of zeolite and 50.7 g of Pural SB were weighed in, mixed and etched with formic acid, and the mixture was processed with 38 ml of water to give a homogeneous material. The kneaded material was forced with the aid of an extrudate press through a 2.5 mm die at approx. 110 bar. Subsequently, these extrudates were dried in a drying cabinet at 120° C. for 16 h, calcined in a muffle furnace at 500° C. (heating time 4 h) for 4 h and processed in a sieving machine with 2 steel balls (diameter approx. 2 cm, 258 g/ball) to give 1.6-2 mm spall. The BET surface area of the resulting spall was 291 m²/g.

Elemental Analysis:
Si: 24.5 g/100 g
Al: 19.0 g/100 g
Mg: 2.3 g/100 g
Na: 0.04 g/100 g

### Reference Example 9

### Process for Converting a Gas Stream Comprising dimethyl ether and CO₂ to olefins

The catalysts prepared in reference examples 7 and 8 (in each case 2 g) were mixed with silicon carbide (in each case 23 g) and installed in a continuously operated, electrically heated tubular reactor. The dimethyl ether/CO₂ feed was mixed with nitrogen in a ratio (% by vol.) of dimethyl ether: CO₂:N₂ of 35:35:30 and fed directly into the reactor. In the experiments, the gas stream was converted at a temperature of 450 to 500° C., a loading of 2.2 g of carbon per gram of catalyst and hour (2.2 g C×g_{catalyst}⁻¹×h⁻¹) based on dimethyl ether and at an (absolute) pressure of 1 to 2 bar, with maintenance of the reaction parameters over the entire run time. Downstream of the tubular reactor, the gaseous product mixture was analyzed by on-line chromatography.

The results achieved in the test reactor for the catalysts according to reference examples 7 and 8 with respect to the selectivities are shown in table 3, these reproducing the average selectivities during the run time of the catalyst in which the conversion of dimethyl ether was 95% or more.

TABLE 3
Average selectivities at a dimethyl ether conversion of >95%.

| Product | Reference ex. 7 | Reference ex. 8 |
|---|---|---|
| ethylene | 10 | 8 |
| propylene | 29 | 32 |
| butylene | 22 | 26 |
| C₄ paraffins | 6 | 4 |
| C₅₊ (mixture) | 19 | 22 |
| aromatics | 10 | 6 |
| C₁-C₃ paraffins | 4 | 2 |

1.-32. (canceled)

33. A process for converting a gas mixture comprising CO and H₂ to olefins, comprising
(1) providing a gas mixture (G0) comprising CO and H₂;
(2) providing a catalyst (C1) for conversion of CO and H₂ to dimethyl ether;
(3) contacting the gas mixture (G0) with the catalyst (C1) to obtain a gas mixture (G1) comprising dimethyl ether and CO₂;
(4) providing a catalyst (C2) for conversion of dimethyl ether to olefins;
(5) contacting the gas mixture (G1) comprising dimethyl ether with the catalyst (C2) to obtain an olefin-comprising gas mixture (G2).

34. The process according to claim 33, wherein the CO₂ present in gas mixture (G1) is fully or partly removed between steps (3) and (5).

35. The process according to claim 34, wherein only CO₂ is fully or partly removed from gas mixture (G1) between steps (3) and (5).

36. The process according to claim 33, wherein no CO₂ is removed from gas mixture (G1) between steps (3) and (5).

37. The process according to claim 36, wherein no components are removed from gas mixture (G1) nor are any further gas streams supplied thereto between steps (3) and (5).

38. The process according to claim 33, wherein the gas mixture (G1) which is contacted with (C2) in (5) has a CO₂ content in the range from 20 to 70% by volume based on the total volume of the gas mixture.

39. The process according to claim 33, wherein the module according to formula (I):
$$\frac{{H}_{2} [ \% by vol . ] - {CO}_{2} [ \% by vol . ]}{CO [ \% by vol . ] + {CO}_{2} [ \% by vol . ]} \qquad ( I )$$
for the gas mixture (G0) is in the range from 5:95 to 66:34.

40. The process according to claim 33, wherein the gas mixture (G1) which is contacted with (C2) in (5) has a content of dimethyl ether in the range from 20 to 70% by volume based on the total volume of the gas mixture.

41. The process according to claim 33, wherein the molar CO₂:dimethyl ether ratio in (5) of the gas mixture (G1) which is contacted with (C2) in (5) is in the range from 10:90 to 90:10.

42. The process according to claim 33, wherein the gas mixture (G1) which is contacted with (C2) in (5) has an H₂ content in the range from 0 to 35% by volume based on the total volume of the gas mixture.

43. The process according to claim 33, wherein the molar H₂:dimethyl ether ratio in (5) of the gas mixture (G1) which is contacted with (C2) in (5) is in the range from 0 to 64:36.

44. The process according to claim 33, wherein the gas mixture (G1) which is contacted with (C2) in (5) has a content of methanol in the range from 0 to 20% by volume based on the total volume of the gas mixture.

45. The process according to claim 33, wherein the molar methanol:dimethyl ether ratio in (5) of the gas mixture (G1) which is contacted with (C2) in (5) is in the range from 0.1:99.9 to 50:50.

46. The process according to claim 33, wherein the gas mixture (G1) which is contacted with (C2) in (5) has an H₂O content in the range from 0 to 20% by volume based on the total volume of the gas mixture.

47. The process according to claim 33, wherein the molar H₂O: dimethyl ether ratio in (5) of the gas mixture (G1) which is contacted with (C2) in (5) is in the range from 0 to 22:78.

48. The process according to claim 33, wherein the provision of gas mixture (G0) in (1) comprises the obtaining of the gas mixture from a carbon source.

49. The process according to claim 48, wherein the provision of gas mixture (G0) comprises the conversion of carbon or hydrocarbon to a product comprising hydrogen and carbon monoxide.

50. The process according to claim 33, wherein the contacting in (3) is effected at a temperature in the range from 150 to 400° C., preferably from 200 to 300° C.

51. The process according to claim 33, wherein the contacting in (3) is effected at a pressure in the range from 2 to 150 bar, preferably from 20 to 70 bar, more preferably from 30 to 50 bar.

52. The process according to claim 33, wherein the contacting in (5) is effected at a temperature in the range from 150 to 800° C.

53. The process according to claim 33, wherein the contacting in (5) is effected at a pressure in the range from 0.1 to 20 bar.

54. The process according to claim 33, wherein at least part of the process is performed continuously.

55. The process according to claim 54, in which the space velocity in the contacting in (3) is in the range from 50 to 50 000 h⁻¹.

56. The process according to claim 54, in which the space velocity in the contacting in (5) is in the range from 0.3 to 50 h⁻¹, preferably in the range from 0.5 to 40 h⁻¹, further preferably from 1 to 30 h⁻¹.

57. The process according to claim 33, wherein catalyst (C1) comprises one or more catalytically active substances for conversion of synthesis gas to methanol; and
one or more catalytically active substances for dehydration of methanol.

58. The process according to claim 57, wherein the one or more catalytically active substances for conversion of synthesis gas to methanol are selected from the group consisting of copper oxide, aluminum oxide, zinc oxide, ternary oxides and mixtures of two or more thereof.

59. The process according to claim 57, wherein the one or more catalytically active substances for dehydration of methanol are selected from the group consisting of aluminum hydroxide, aluminum oxide hydroxide, gamma-aluminum oxide, aluminosilicates, zeolites and mixtures of two or more thereof.

60. The process according to claim 57, wherein the one or more catalytically active substances for dehydration of methanol are doped with niobium, tantalum, phosphorus and/or boron.

61. The process according to claim 33, wherein catalyst (C2) comprises one or more zeolites of the MFI, MEL and/or MWW structure type and particles of one or more metal oxides, the one or more zeolites preferably being of the MFI structure type.

62. The process according to claim 61, wherein the one or more zeolites of the MFI, MEL and/or MWW structure type comprise one or more alkaline earth metals, preferably Mg.

63. The process according to claim 61, wherein the one or more zeolites of the MFI, MEL and/or MWW structure type comprise phosphorus, the phosphorus being present at least partly in oxidic form.

64. The process according to claim 61, wherein the particles of the one or more metal oxides comprise phosphorus, the phosphorus being present at least partly in oxidic form.
